(19) United States
(12) Patent Application Publication
(10) Pub. No.: US 2023/0357807 A1
(43) Pub. Date: Nov. 9, 2023

(54) CATALYTICALLY ACTIVE PROTEIN FOAMS, AND METHOD FOR PRODUCING SAME

(71) Applicant: Karlsruher Institut fuer Technologie, Karlsruhe (DE)

(72) Inventors: Christof Niemeyer, Bremen (DE); Kersten Rabe, Karlsruhe (DE); Maximilian Groesche, Karlsruhe (DE); Patrick Bitterwolf, Karlsruhe (DE); Laurenz Walkowsky, Karlsruhe (DE)

(21) Appl. No.: 18/006,728

(22) PCT Filed: Jun. 25, 2021
(86) PCT No.: PCT/EP2021/025233

§ 371 Date: Jan. 25, 2023

(30) Foreign Application Priority Data
Jul. 27, 2020 (DE) 10 2020 119 698.9

(51) Int. Cl. C12N 9/10; C12N 9/88; C12P 13/00; C12N 11/14; C12N 9/04; C12P 7/22; C12N 11/18; C12N 9/06

(52) U.S. Cl. CPC C12P 13/008; C12N 9/0006; C12N 9/0028; C12N 9/1096; C12N 9/88; C12N 11/14; C12N 11/18; C12P 7/22; C12Y 101/01001; C12Y 101/01047; C12Y 105/01; C12Y 206/01029; C12Y 401/01

(57) ABSTRACT

Catalytically active protein foams and methods for producing same by coupling catalytically active fusion proteins with connectors, and the use of catalytically active protein foams in biocatalysis and microfluidics.

### CROSS-REFERENCE TO PRIOR APPLICATIONS

This application is a U.S. National Phase application under 35 U.S.C. § 371 of International Application No. PCT/EP2021/02533, filed on Jun. 25, 2021, and claims benefit to German Patent Application No. 10 2020 119 698.9, filed on Jul. 27, 2020. The International Application was published in German on Feb. 3, 2022 as WO 2022/022850 A1 under PCT Article 21(2).

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: 23,031 bytes ASCII (Text) file named “819259_ST25,” created Jan. 19, 2023.

### FIELD

The present invention relates to catalytically active protein foams and methods for the production thereof.

### BACKGROUND

Biocatalysis refers to a reaction and acceleration or control of chemical reactions in which enzymes serve as biological catalysts. Enzymes consist completely or predominantly of one or more proteins and partially also of a co-factor.

Biocatalysis offers several advantages over the conventional chemical catalysis: First, the reactions are typically carried out in a milder temperature range (4-60° C.), which leads to a lower energy requirement required for the reactions. Enzyme reactions can be carried out in an aqueous environment; in parallel thereto, the use of enzymes in combination with organic solvents has increased in the last few decades. Biocatalytic reactions in two-phase systems and in pure organic solvents enable higher substrate loading, prevent hydrolysis of water-sensitive compounds and shift the thermodynamic equilibrium of many reactions. A further advantage of biocatalytic reactions is that they do not have to be carried out under protective gas, while many homogeneous and heterogeneous catalysts are oxidized and deactivated by air already at room temperature (E. M. M. Abdelraheem et al., React. Chem. Eng. 2019, 4, 1878-1894).

Due to the complex but defined 3-dimensional structure of enzymes, enzyme catalysis benefits from a high chemo-, regio- and stereoselectivity, which allows the production of complex and chiral molecules. Nowadays, biocatalysis is used for a series of industrial processes, such as for example for the production of valuable fine chemicals, optically active pharmaceuticals, crop protection products and fragrances or for the degradation of plastics. Since enzymes are less harmful to health and the environment than chemical catalysts, their use is favored in the food and beverage industry (E. M. M. Abdelraheem et al., React. Chem. Eng. 2019, 4, 1878-1894).

Biocatalysis offers the possibility to redesign entire synthesis routes for the production of important molecules, to obtain them in higher yields and to simplify their downstream processes. The advantages of synthesis strategies based on organic one-pot transformations without having to isolate intermediate compounds are known from numerous studies. Such processes are also referred to as “cascade” reactions (E. M. M. Abdelraheem et al., React. Chem. Eng. 2019, 4, 1878-1894).

Protein technology makes it possible to optimize enzymes by changing their amino acid sequence with respect to expression level, stability, catalytic activity and selectivity, and to adapt them to industrial requirements. For example, new variants of the P450 enzymes with desired activity have been generated, which enable a remarkable variety of chemical reactions, such as the regiospecific oxyfunctionalization, C-C bond formation, cyclopropanation, etc. Lipases were also subjected to protein engineering studies which have led to new catalysts for the kinetic chiral resolution of chiral alcohols (E. M. M. Abdelraheem et al., React. Chem. Eng. 2019, 4, 1878-1894).

Enzyme immobilization is a comparatively old approach to make enzymes technically usable (N. Grubhofer et al., Naturwissenschaften 1953, 40, 508). Today, immobilized enzymes are used extensively, inter alia in production methods of chemistry and food industry, in diagnostics and biosensing or in washing agents, agricultural products and cosmetics.

Enzyme immobilization is generally defined as the “physical differentiation or localization of enzymes in a limited space to obtain the catalytic activity” (E. Katchalski-Katzir, Trends Biotechnol. 1993, 11, 471-478). Thus, by immobilization, a concentration of the enzymes and thus a higher productivity (space-time yield) can be achieved and the amount of catalyst used can be reduced. Depending on the choice of the immobilization strategy, the stability to high temperatures, extreme pH values, mechanical shear forces and organic solvents can be changed. At the same time, the separation of the enzymes from the reaction medium is facilitated, thereby enabling the use of the enzymes in several reaction cycles. In addition, immobilization can be accompanied by an intrinsic stabilization of the enzymes, so that storability and process life are significantly improved compared to the free enzyme (R. A. Sheldon et al., Chem Rev 2018, 118, 2, 801-838).

In principle, the covalent and non-covalent carrier binding, inclusion process in polymeric matrices and in membranes and carrier-free immobilization can be distinguished as immobilization methods. Conventional methods for fixing enzymes are based on the anchoring or cross-linking of the proteins by polyfunctional coupling reagents (“crosslinker”), either directly in the channel structure or on porous carrier materials (polymers, particles). The use of carrier materials here represents a compromise which, although it can enable effective immobilization to obtain the catalytic activity, also inevitably leads to “dilution” of the catalytic species and thus to the reduction of space/time yields. Carrier-free insoluble aggregates (immobilizates) of enzymes are formed by direct or via spacer-mediated covalent cross-linking of the individual protein molecules (U. Rössl et al. Biotechnol. Lett. 2010, 32, 341-350). In this case, bifunctional reagents are used which react with the reactive functions of the amino acid side chains on the enzyme surface. As in the case of covalent carrier binding, the ε-amino functions of the lysine residues primarily serve as reactants. Suitable cross-linking reagents are dialdehydes (e.g., glutardialdehyde, dextran dialdehyde), diisocyanates (e.g., hexamethyldiisocyanate) and diisothiocyanates (e.g., p-phenylene diisothiocyanate). Cross-linking is possible both on the basis of dissolved enzymes and also on the basis of freeze-dried or spray-dried preparations, enzyme crystals and pre-aggregated enzymes. In particular, cross-linked enzyme crystals (CLEC) and cross-linked enzyme aggregates (CLEA) have become important for application technology. CLECs have a solid microporous structure with uniform channels passing through the crystal. The particle diameter can be adjusted flexibly to between 1 and 100 µm via the ratio of enzyme and cross-linking reagent or the incubation time.

The generation of CLEAs makes use of the spontaneous agglomeration and precipitation of proteins in the presence of various organic solvents, electrolytes, non-ionic polymers and acids and takes place by obtaining the three-dimensional structure, i.e., without denaturation. The resulting aggregates are cross-linked to give particles of gel-like consistency with a diameter of 5-50 µm. (Einführung in die Enzymtechnologie, K.-E. Jaeger et al., 2018 Springer Spektrum, Springer-Verlag GmbH Deutschland, 187-206; R. A. Sheldon, Org. Process Res. Dev. 2011, 15, 1, 213-223).

A solution for loading with relatively large amounts of active biocatalysts is provided by the in-situ generation of pure enzyme hydrogels. Hydrogels are porous polymers produced from natural or synthetic structural proteins and explored within the biomedicine for the encapsulation of cells and tissue engineering (J. H. Lee, J. Tissue Eng. 2018, 9, 1-4).

A recently introduced protein gel formation strategy uses a pair of genetically encoded reactive partners, SpyTag and SpyCatcher, which spontaneously undergo a covalent isopeptide bond under physiological conditions (S. C. Reddington et al., Current Opinion in Chemical Biology 2015, 29, 94-99; EP2534484B1). Peschke et al. describe a method for producing self-assembling enzyme hydrogels in which enzymes are genetically fused to a SpyTag or SpyCatcher domain. These enzymes fused to connectors in this way cross-link in situ with themselves and form monolithic (wholly polymerized) “all enzyme hydrogels”. The cross-linked polymers thus produced typically have pore sizes of < 200 nm and show a higher catalytic activity than the previously mentioned CLEAs.

Enzyme immobilization has also been described in the form of foams: WO9820100A1 describes non-cross-linked enzyme formulations in detergents which are subsequently foamed. However, extremely low enzyme concentrations in the range of 0.0001-0.005% are used in these. US4195127A describes the production of enzyme-containing polyurethane-based biohybrid foams, and Brun et al. describe the production of mesoporous silica-based biohybrid foams, on which enzymes are subsequently immobilized (N. Brun et al., Chem. Mater. 2010, 22, 16, 4555-4562).

The production of foams is an established technology which is based on introducing bubbles into a liquid phase (W. Drenckhan et al., Advances in Colloid and Interface Science 2015, 222, 228-259; W. Drenckhan et al., Advances in Colloid and Interface Science 2015, 224, 1-16).

Foams can be produced, for example, by passing gas through a membrane (J. Dittmann et al., Journal of Colloid and Interface Science 2016, 467, 148-157) or by shaking a container containing a liquid admixed with a surfactant (A. Maestro et al., Soft Matter 2014, 10, 36, 6975-6983).

The microfluidic production of foams is based on the generation of individual compartments in two-phase systems due to shearing from one phase into the other. The two phases are not miscible. This technology is known as droplet microfluidics (S. Mashagi et al., Trends in Analytical Chemistry 2016, 82, 118-125). The literature distinguishes between droplets (liquid-liquid systems) and bubbles (liquid-air systems), wherein the generation of bubbles can take place analogously to generation of droplets in different geometries (S. L. Anna, Annu. Rev Fluid. Mech. 2016, 48, 285-309). The established methods are the T intersection and the flow-focusing intersection (T. Fu et al., Chemical Engineering Science 2015, 135, 343-372).

The bubble systems are further distinguished in whether the gas phase is used as a continuous phase (P. Tirandazi et al., Journal of Micromechanics and Microengineering 2017, 27, 075020) or as a disperse phase (S. Andrieux et al., Langmuir 2018, 34, 1581-1590). These bubble systems differ with respect to the flow regime and the bubble stabilization systems. The bubbles thus generated have bubble sizes in the range from 10 µm to 1000 µm and fuse in a self-assembly process into higher-level grating structures, which can then be changed in a controlled manner via the flow parameters. Depending on the external geometry parameters of the structure, these gratings form dynamically assembled foams (P. Garstecki, Applied Physics Letters 2004, 85, 13, 2649-2651).

Protein foams are an essential component of the food industry and are widely used for the uniform insertion of fine air cells for the texturing of foodstuffs. In this case, catalytically inactive mixtures of structural proteins such as, for example, egg white, gelatin, soy or whey proteins are used for generating foam.

When forming a protein foam, a distinction is made between three phases (J. R. Clarkson et al., Journal of Colloid and Interface Science 1999, 215, 2, 323-332):

Phase 1: The proteins diffuse to the air/water interface, are thereby concentrated locally and thus reduce the surface tension. Phase 2: The proteins adsorbed at the interface develop partially, with orientation of their hydrophilic polar fractions to the aqueous phase and of their hydrophobic fractions to the non-aqueous phase.

Phase 3: In addition, the proteins interact with one another at the interface and form a film which can lead to further partial development and coagulation. Via the three phases mentioned, proteins adsorb rapidly at the interface and form a stabilizing film around individual bubbles. This promotes foam formation.

In the so-called foam fractionation of proteins, the concentration of the surface-active proteins at the interface is used for fractional separation. In this case, an inert gas flows through a protein-containing solution and the resultant foam is successively separated off. This process enables a cost-effective and simple method to separate proteins from a solution and is suitable for industrial purification of enzymes (F. Uraizee et al., Enzyme Microb. Technol. 1990, 12, 315-316).

However, due to the denaturation of proteins at the interfaces, foams generally act in a destabilizing manner in connection with catalytically active proteins and inactivate them substantially (J. R. Clarkson et al., Journal of Colloid and Interface Science 1999, 215, 2, 333-338; Z. Liu et al., Bioseparation 1998, 7, 3, 167-174).

Continuous flow biocatalysis is an emerging area of industrial biotechnology that uses enzymes immobilized in flow channels for the production of higher-quality chemicals. Biocatalytic flow processes are difficult to realize because the heterogeneous catalysis system requires effective surface immobilization techniques that are more demanding for enzymes than for conventional organo(metallic) catalysts.

Common methods for enzyme immobilization within microstructured flow channels such as physisorption, chemical cross-linking or genetically encoded immobilization tags have proven their applicability, but the problem remains that the amount of the immobilized biocatalyst is limited by the effective surface. To overcome this restriction, pseudo-3D interface layers of synthetic polymers or micro-/nanoparticles may be used to increase the loading capacity for enzymes. However, these approaches waste the limited reactor space and also often require additional coupling steps with possible disadvantages for the biocatalytic activity. One alternative are the aforementioned self-assembling “all-enzyme hydrogels” which have relatively high space-time yields in biocatalytic flow processes as immobilized biocatalysts (T. Peschke et al., Angew. Chem. Int. Ed. 2018, 57, 17028-17032).

Catalytically active protein foams produced from catalytically active proteins that are coupled to one another have not yet been described.

Also, the use of catalytically active protein foams as a carrier-free material for flow biocatalysis has not been known until now.

### SUMMARY

In an embodiment, the present invention provides a method for producing catalytically active protein foams, the method comprising the steps of:
A) Producing catalytically active proteins fused to a first connector (7) and catalytically active proteins fused to a second connector (8) that is complementary to the first connector (7);
B) Producing bubbles containing catalytically active proteins in a bubble generator using a gas phase and a solution of the catalytically active proteins fused to the first connector (7) and the catalytically active proteins fused to the connector second (8) from step A);
C) Transferring the bubbles produced in step B) into a reaction chamber (5);
D) Coupling the catalytically active proteins contained in the bubbles from step B) in the reaction chamber (5) to form a catalytically active protein foam (10).

### BRIEF DESCRIPTION OF THE DRAWINGS

The invention is explained in more detail with reference to the following figures, exemplary embodiments and descriptions.

All features illustrated and their combinations are not limited to only these figures and exemplary embodiments and their designs. Rather, they should be regarded as being combinable with other possible designs that are feasible, but not explicitly illustrated as exemplary embodiments.

FIG. 1 shows the schematic representation of a bubble generator for producing bubbles containing catalytically active proteins (6).

FIG. 2 shows the schematic representation of the reaction chamber (5) for the flow-catalytic processing of the protein foam. FIG. 2a) shows an isometric view of the reaction chamber. FIG. 2b) shows the side view and FIG. 2c) shows the top view of the structure.

FIG. 3 schematically shows the entire process of producing a catalytically active protein foam (10).

FIG. 4 shows high-speed recordings of the reproducible generation of monodisperse bubbles, which were produced by means of the bubble generator from FIG. 1, and their spatial arrangement in the outlet channel (4), depending on the pressures applied.

In FIG. 5, the time-dependent cross-linking of the protein foam is documented by transmitted light microscopy. FIG. 5a) shows the protein foam in the reactor chamber after 12 minutes. The bubbles arrange themselves as hexagonally densely packed Kepler FCC (face centered cubic) sphere packings. FIG. 5b) shows an intermediate drying step after 60 minutes. After drying and thus reduction of the liquid fraction, the sphere packing transitions into an arrangement after about 2 hours which is similar to a Kelvin BCC (body-centered cubic) packing (FIG. 5c)).

FIG. 6 shows a fluorescence microscopic image of the interfaces of the dried protein foam matrix.

FIG. 7 shows an image of the catalytically active protein foam (10) with a confocal laser scanning microscope. On the top layer of the protein foams, intact thin films have formed, inter alia. Dark regions show the lamellae between the bubbles, light regions the outer side of the bubbles covered with a cross-linked protein thin film (FIG. 7a)). FIG. 7b) shows a detail of a bubble in which this thin film is perforated due to external forces. FIG. 7c) shows a closed, intact thin film.

FIG. 8 illustrates the reactor performance of various, catalytically active protein foam systems.

FIG. 9 shows the stability of the catalytically active protein foam (10) on the basis of the relative foam height in a cuvette with and without addition of additives over a period of > 84 h. 35.4 µM saponin was used as the additive.

FIG. 10 shows the catalytic conversion of the catalytically active protein foam (10) after 48 h. Here, the protein foams (10) stabilized by means of surfactant have a 20% higher catalytic conversion than the non-stabilized protein foams (10).

FIG. 11 shows examples of the production of the catalytically active protein foams (10) which consist of the enzymes LbADH/BsGDH, which are genetically fused to the non-covalent protein/protein interaction domains SH3, PDZ and GBD and their corresponding ligands.

FIG. 12 shows the catalytic conversion at the time t =30 h of flow bioreactors as a function of the drying duration using the enzyme system LbADH/BsGDH, which is linked either by means of the covalent connector pair SC/ST or by means of the non-covalent connector pair of the protein/protein interaction domain SH3 and the corresponding ligand.

### DETAILED DESCRIPTION

Embodiments of the present invention provide catalytically active protein foams and a method for the production thereof, wherein the protein denaturation during foam formation is to be as low as possible, but the volume-specific enzyme loading and the stability of the protein foams should be as high as possible.

Embodiments of the invention produce catalytically active protein foams which consist essentially solely of catalytically active proteins coupled to one another or of composite materials including the catalytically active proteins coupled to one another. In certain embodiments, the proteins are an essential constituent of the material itself, i.e., they are not applied subsequently to foamed materials, such as, for example, polymers.

In the following, the terms “protein foam” and “protein foams” are used synonymously and are defined as gaseous bubbles enclosed by liquid or solid protein thin films.

The method according to certain embodiments of the invention comprises the following steps:
A) Producing catalytically active proteins fused to a connector (7) and catalytically active proteins fused to a connector (8) that is complementary to the connector (7);
B) Producing bubbles containing catalytically active proteins in a bubble generator using a gas phase and a solution of the catalytically active proteins fused to the connector (7) and the catalytically active proteins fused to the connector (8) from step A);
C) Transferring the bubbles produced in step B) into a reaction chamber (5);
D) Coupling the catalytically active proteins contained in the bubbles from step B) in the reaction chamber (5) to form a catalytically active protein foam (10).

One feature of embodiments of the catalytically active protein foam (10) is that the individual molecules of the catalytically active proteins (6) are coupled to one another via connectors (7) and (8) that are complementary to one another. “Coupled to one another” means for the method according to embodiments of the invention that the connectors (7) and (8) enter into a covalent or non-covalent bond. In the method according to embodiments of the invention, non-covalent bonds are, for example, hydrogen bonds or ionic bonds, but also hydrophobic interactions and van der Waals forces.

In the method according to an embodiment of the invention, complementary to one another means for the connectors (7) and (8) that they react specifically with one another so that a specific bond is formed between the catalytically active proteins. In one embodiment, the bond is a covalent bond, in another embodiment a non-covalent bond.

For the SpyTag/SpyCatcher system, the complementarity consists in that the Nε of a lysine residue of the SpyCatcher domain specifically reacts with the C_{α} of a (complementary) aspartic acid residue of the SpyTag domain to form an isopeptide bond and cleavage of H₂O (S. C. Reddington et al., Current Opinion in Chemical Biology 2015, 29, 94-99).

In one embodiment, the catalytically active proteins fused to a connector (7) and the catalytically active proteins fused to the connector (8) complementary to the connector (7) are expressed heterologously in step A), and the connectors are a genetically fused part of the proteins. For the heterologous expression of the catalytically active proteins fused to the connectors (7) or (8), bacterial cells are transformed with the corresponding expression vector.

In one embodiment, the connectors are genetically fused peptide tags which can form covalent (iso)peptide bonds, such as, for example, split inteins (N. H. Shah et al., Chem Sci 2013, 5, 446-461), sortases (H. Mao et al., JACS 2004, 126, 2670-2671), transglutaminases (A. Fontana et al., Adv Drug Deliv Rev 2008, 60, 13-28), peptiligases (A. Toplak et al., Adv Synth Catal. 2016, 358(13), 2140-2147) or the SpyTag/SpyCatcher system.

For example, the catalytically active protein (6) is genetically fused to a SpyTag or SpyCatcher domain. The reaction of the SpyTag with the SpyCatcher leads to a defined covalent bond between the catalytically active proteins.

In a further embodiment, genetically fused, non-covalent, peptide or protein-based bond systems are used for linking the catalytically active proteins (6), such as, for example, the RIDD/RIAD system (W. Kang et al., Nat. Commun. 2019, 10, 4248), self-assembling amphipathic peptides (W. Zhao et al., Microb Cell Fact. 2019, 18, 91), leucine-zipper systems (J. R. Moll et al., Protein Sci. 2001, 10, 3, 649-655) or elastin-like peptides (ELPs) and collagen-like peptides (CLPs) (D. W. Urry et al., Biopolymers. 1985, 24, 12, 2345-2356; T. Luo et al., J. Am. Chem. Soc. 2015, 137, 49, 15362-15365).

One particular embodiment are the protein/protein interaction domains PDZ (PSD95-Discs large _ZO1), GBD (GTPase-binding domain), SH3 (Src-homology 3) and their corresponding ligands (J. Dueber et al., Nat. Biotechnol. 2009, 27, 753-759). In a further particular embodiment, so-called single domain antibodies and their corresponding antigens are used (R. H. J. van der Linden et al., Biochimica et Biophysica Acta 1999, 1431, 37-46). In a further particular embodiment, so-called macrocyclic host-guest interactions are used to link the catalytically active proteins (6). An exemplary embodiment is the use of an FGG (phenyalanine-glycine-glycine) peptide motif fused to the catalytically active proteins and the use of cucurbit[8]uril (D. H. Nguyen et al., Angew Chem Int Edit. 2010, 49, 5, 895-898).

In another embodiment, catalytically active proteins (6) are expressed heterologously in step A), bacterial cells being transformed with the corresponding expression vector for the heterologous expression. In contrast, the connectors (7) or (8) are subsequently chemically bound to the catalytically active proteins (6). In a preferred embodiment, the connectors are hetero- or homobifunctional crosslinkers for covalent linking of the catalytically active proteins (6), for example sulfosuccinimidyl 4-(N-maleimidomethyl)cyclohexane-1-carboxylate,n-succinimidyl 3-(2-pyridyldithio) propionate, dimethyl adipimidate or dialdehydes, such as, for example, 1,5-pentandial. Crosslinkers having two identical reactive groups are referred to as homobifunctional crosslinkers, and crosslinkers having two different groups are referred to as heterobifunctional crosslinkers.

In a preferred embodiment, the catalytically active protein (6) is an enzyme, for example a stereoselective enzyme and/or an enzyme selected from the group of oxidoreductases (EC 1.-.-.-), transferases (EC 2.-.-.-), hydrolases (EC 3.-.-.-), lyases (EC 4.-.-.-), isomerases (EC 5.-.-.-), ligases (EC 6.-.-.-) and translocases (EC 7.-.-.-). The number of the Enzyme Commission (EC number) is a numerical classification scheme for enzymes based on the chemical reactions catalyzed by them. (https://www.enzyme-database.org/).

Examples of enzymes used in the method according to embodiments of the invention are Lactobacillus brevis alcohol dehydrogenase (LbADH), Bacillus subtilis glucose 1-dehydro-genase (BsGDH), Enterobacter sp. phenacrylate decarboxylase (EsPAD), Saccharomyces cerevisiae NADP(H)-dependent alcohol dehydrogenase (Gre2P), Streptomnyces sp. (S)-imine reductase (GF3546), Halomonas elongata amine transaminase (HEWT).

In the method according to embodiments of the invention, the catalytically active protein foams (10) comprise a catalytically active protein (6) or a plurality of catalytically active proteins (6) with different reaction specificities.

In one embodiment for producing a monoenzyme foam, the catalytically active protein (6), which is fused to the connector (7), is identical to the catalytically active protein (6), which is fused to the connector (8). In the method according to embodiments of the invention, monoenzyme foam means that the catalytically active protein foam (10) contains only one catalytically active protein (6).

In one embodiment for producing a bi-enzyme foam, different catalytically active proteins (6) are linked to one another in step D) via the connectors (7) and (8), in that in step A) a catalytically active protein (6) is fused to the connector (7) and a catalytically active protein (6) different therefrom is fused to the connector (8) complementary to the connector (7) (method a). In the method according to embodiments of the invention, bi-enzyme foam means that the catalytically active protein foam (10) contains two different catalytically active proteins (6).

In a further embodiment for producing a bi-enzyme foam or a multi-enzyme foam, a plurality of mutually orthogonal connector pairs are used (method b). In the method according to embodiments of the invention, multi-enzyme foam means that the catalytically active protein foam (10) contains at least three different catalytically active proteins (6).

In the method according to embodiments of the invention, orthogonal connector pairs means that a first connector pair comprising the associated connectors (7) and (8) is specifically coupled to one another, and that at least one second connector pair comprising the respective associated connectors (7) and (8), likewise is coupled specifically to one another without a cross coupling occurring of the first and the at least second connector pair. Brune et al. describe, for example, two orthogonal connector pairs SpyTag/SpyCatcher and SnoopTag/SnoopCatcher, which are used to produce synthetic nanoparticles with different antigens. In this case, the SpyCatcher domain forms an isopeptide bond with the SpyTag domain by spontaneous amidation, whereas the SnoopCatcher domain forms an isopeptide bond with the SnoopTag domain by a transamidation (K. D. Brune et al., Bioconjugate Chem. 2017, 28, 1544-1551; D. Hatlem et al., Int. J. Mol. Sci. 2019, 20, 2129).

To produce a bi-enzyme foam using two orthogonal connector pairs, a catalytically active protein (6) is coupled to one another via a first connector pair comprising the associated connectors (7) and (8), and a second catalytically active protein (6) is coupled to one another via a second connector pair orthogonal to the first connector pair, comprising the respective associated connectors (7) and (8) (method b).

To produce a multi-enzyme foam using at least three orthogonal connector pairs, at least three different catalytically active proteins (6) are fused to the respective connector (7) or (8), wherein the respective connectors (7) and (8) each form a connector pair. For example, to produce a multi-enzyme foam containing three different catalytically active proteins (6), a first catalytically active protein (6) is coupled to one another via a first connector pair comprising the associated connectors (7) and (8), a second catalytically active protein (6) is coupled to one another via a second connector pair orthogonal to the first connector pair, comprising the respective associated connectors (7) and (8), and a third catalytically active protein (6) is coupled to one another via a third connector pair orthogonal to the first and second connector pairs, comprising the respective associated connectors (7) and (8). For the production of a multi-enzyme foam containing at least 4 different catalytically active proteins (6), further orthogonal connector pairs are accordingly used according to the method according to embodiments of the invention.

It is also conceivable to produce a multi-enzyme foam by combining methods a) and b), i.e., a heterodimer of catalytically active proteins (6) is formed via a first connector pair, and an at least second heterodimer of catalytically active proteins (6) is formed via an at least second connector pair orthogonal thereto.

In a further step B), the method according to embodiments of the invention comprises the production of bubbles comprising catalytically active proteins. These bubbles are produced in a fluidic device in the method according to embodiments of the invention.

In one embodiment, the fluidic device comprises a bubble generator. For the production of the bubble generator, various materials such as, for example, metals, glass or polymers are used in the method according to the invention. Polydimethylsiloxane and polymethyl methacrylate are preferably used for the production of the bubble generator. In order to use the structures as fluidic units, the structures must be closed. The structure is bonded either by thermal plasma oxidation to a glass microscope slide or to polydimethylsiloxane. Alternatively, an adhesive film for sealing (for example made of polyolefin) is used.

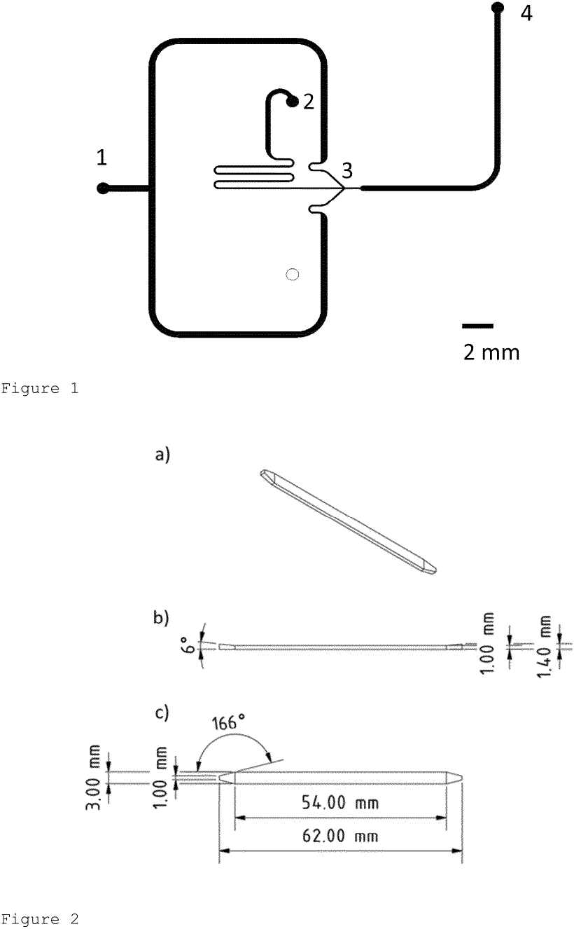

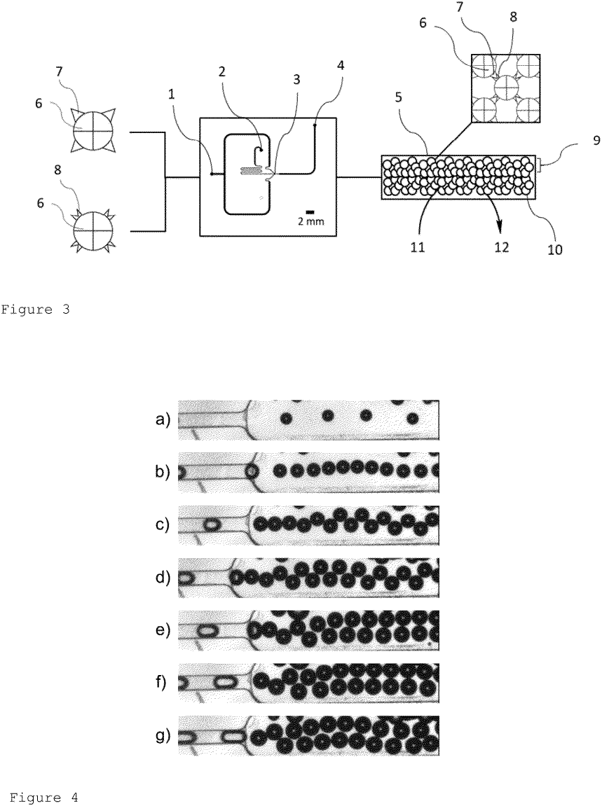

The first element of the bubble generator (FIG. 1) is an inlet (1) for a continuous phase which splits into two mirror-symmetrical paths. These impinge on an inlet channel for a disperse phase (2) at a 45° angle from both sides. The latter corresponds to the gas of which the bubbles consist. The channel cross-section can have any profile. The channel of the disperse phase intersects the channel cross-section of the continuous phase in its mirror axis and thus generates the functional element of the flow-focusing intersection (3). Both phases meet at the intersection (3) on which the gas phase is separated into individual bubbles by the continuous phase. The bubbles generated at the intersection flow through the outlet channel (4) from the structure into a reaction chamber (5) (FIG. 2). The reaction chamber consists of a stretched rectangular channel which tapers towards the ends in order to ensure a contacting for flow actuation.

In order to generate bubbles in the structure described above, two phases that are not miscible with one another are used. The phase that envelops the bubble is the continuous phase; the phase of which the bubble consists is the disperse phase. All combinations of gas/liquid that are difficult to mix with one another can be used as phases for bubble generation.

The gas phase used in the method according to embodiments of the invention is any gas, preferably an inert gas. In a particularly preferred embodiment, nitrogen is used as the gas phase.

In the method according to embodiments of the invention, a solution of catalytically active proteins fused to connectors is used as the continuous phase.

In one embodiment, the solution of the catalytically active proteins fused to connectors is an aqueous solution of the catalytically active proteins fused to connectors. The aqueous solution optionally comprises buffer systems such as, for example, citrate, acetate or phosphate buffer systems. In the method according to embodiments of the invention, an aqueous solution is a solution in which H₂O as solvent is used at > 99.9 volume percent of the solvent fraction. In another embodiment, the solution of the catalytically active proteins fused to connectors also comprises solvent mixtures of H₂O and organic solvents such as, for example, acetonitrile, ethanol, methanol, dimethyl sulfoxide, isopropanol. The volume fraction of the organic solvent in the solvent mixture can be 0.1 - 99.9 volume percent. The solution of catalytically active proteins fused to connectors, which contains such solvent mixtures of H₂O and organic solvents, optionally comprises buffer systems such as, for example, citrate, acetate or phosphate buffer systems.

In another embodiment, organic solvents, for example acetonitrile, ethanol, methanol, dimethyl sulfoxide, isopropanol, which have a water content of less than 0.1 percent by volume, are used for the solution of the catalytically active proteins fused to connectors.

In a further embodiment, non-classical solvents can be used for the solution of the catalytically active proteins fused to connectors, for example ionic liquids, low-melting eutectic solvents or super-critical CO₂.

In the method according to embodiments of the invention, the concentration of the solution of catalytically active proteins fused to connectors is between 1 µM and 100 mM, depending on the desired polymerization time. Preferred is a solution of catalytically active proteins fused to connectors with a concentration of 1 mM.

In one particular embodiment of the method according to the invention, the protein solutions of the complementary binding partners are mixed with one another in a stoichiometric ratio, incubated and connected to the bubble generator. In this particular embodiment, the bubble generator is first flooded with N₂ gas and constantly flowed through with N₂ gas. Subsequently, the solution of catalytically active proteins fused to connectors is introduced into the microfluidic unit at a constant flow rate and is foamed at the intersection nozzle with the aid of the N₂ gas. The foamed material is collected at the outlet and transferred directly into a reaction chamber (5) (FIG. 2).

The bubbles are stabilized by the proteins used in the method according to embodiments of the invention, which prevents the rapid coalescence of the bubbles.

In one embodiment, additives in small amounts of 10 - 100 µmol/1 are added to the solution of catalytically active proteins fused to connectors. In the method according to embodiments of the invention, additives are substances which have a positive effect on the production of the bubbles in step B) and on the formation of the catalytically active foam (10) in step D). For example, the bubbles in step B) are stabilized over the long term by the addition of additives. Examples of additives in the method according to embodiments of the invention are surfactants, preferably biocompatible surfactants such as, for example, saponins, lauryl sulfobetaines, rhamnolipids, Tween 20 and Triton X100.

The speeds of the continuous and disperse phase are regulated via a pressure gradient Δp. In the method according to embodiments of the invention, the pressure gradient Δp for the continuous phase is between Δp_continuous = 10 mbar and Δp_continuous = 1000 mbar, preferably between 300 and 360 mbar. In the method according to embodiments of the invention, the pressure gradient for the disperse phase is between Δp_disperse = 10 mbar and Δp_disperse = 1000 mbar, preferably between 110 and 200 mbar. It is important that Ap_continuous is greater than Δp_disperse.

The diameter, pore sizes and generation frequency of the bubbles can be precisely adjusted via the geometry of the channels, the pressure gradients used and the type of continuous phase.

With the method according to embodiments of the invention, the bubbles are produced with a diameter of 5-1500 µm and a pore size of 10-2000 µm. In one particular embodiment, bubbles with a diameter of 200-300 µm are generated. The pore sizes of the bubbles thus generated are 50-1200 µm, preferably 250-310 µm.

In another particular embodiment, bubbles with a diameter of 15-200 µm, preferably 30-80 µm, are produced. The pore size of the bubbles is then 15 to 250 µm, preferably 30 to 100 µm.

The bubbles thus produced and containing catalytically active proteins are transferred into a reaction chamber (5). In a preferred embodiment, the reaction chamber (5) consists of polydimethylsiloxane or polymethyl methacrylate.

In the reaction chamber (5), a coupling of the catalytically active proteins takes place via the complementary connectors (7) and (8) used in the method according to embodiments of the invention to form a catalytically active protein foam (10). In the method according to embodiments of the invention, the protein thin film cross-linked in this way on the bubbles of the catalytically active protein foam (10) has a layer thickness of 0.1-200 µm. In one particular embodiment, the layer thickness of the cross-linked protein thin films on the bubbles is 1-60 µm.

In one embodiment, the protein foam is dried in a further step E) after the completed cross-linking reaction in the reaction chamber (5) in step D).

By means of the drying process, the layer thickness of the cross-linked protein thin films on the bubbles can be precisely adjusted to 1-50 µm. Further advantages of drying the catalytically active protein foam (10) are increased mechanical stability, better storability, a higher long-term stability against degeneration processes of the catalytically active proteins contained in the protein foam, and a monodisperse distribution of the pores, and thus a reproducible end product.

In one embodiment, this drying step E) is carried out at room temperature when the reaction chamber (5) is open. In a particular embodiment, drying when the reaction chamber (5) is open is carried out at room temperature with continuous air convection in order to support the drying process.

The drying time when the reaction chamber (5) is open is 30 mins. to 28 days in the method according to embodiments of the invention. Preference is given to a drying time of at least 2 h. Particularly preferred is a drying time of 2-28 days in order to increase the stability and activity of the catalytically active protein foam (10) (FIG. 12).

Another embodiment of drying is to dry the protein foam at 30° C. in an incubator with continuous air convection. The drying time is then 30 mins. to 28 days. In one particular embodiment, the drying time in the incubator is 30-60 mins.

In one particular embodiment of the method according to the invention, the reaction chamber (5) serves as a flow bioreactor by using the catalytically active protein foam (10) dried in step E) to convert at least one substrate (11) to at least one product (12) (FIG. 3). In this case, the at least one substrate (11) is guided as a liquid flow through the reaction chamber (5) at a specific flow rate. After the reaction, the at least one product (12) formed in the reaction chamber (5) is located in the outlet flow of the reaction chamber (5) and is collected at the outlet of the reaction chamber (5).

In another embodiment, the catalytically active protein foams (10) are produced from catalytically active proteins and composite materials, wherein the proteins make up more than 5% (w/w) of the dry material. In the method according to embodiments of the invention, composite materials are materials which consist of two or more components with significantly different physical or chemical properties. In combination, these components produce a material with properties that differ from the individual components, wherein the individual components remain separate and distinguishable within the finished structure (https://www.twi-global.com/technical-knowledge/faqs /what-is-a-composite-material). Examples of such composite materials are DNA-silica nanocomposite materials (Y. Hu et al., Angew. Chem. Int. Ed. 2019, 58, 17269-17272), which consist of a DNA polymer and which are produced by silica nanoparticles (SiNP) with sizes of typically < 200 nm in diameter by a biochemical reaction occurring in homogeneous solution. In a further exemplary embodiment, DNA-SiNP composite materials are used as composite materials, which are adjustable in terms of their mechanical properties by incorporation of carbon nanotubes (CNT) and thus consist of different proportions of DNA, SiNP and CNT (Y. Hu et al., Nat. Commun. 2019, 10, 5522).

In the method according to embodiments of the invention, hybrid materials are produced from the catalytically active proteins and DNA-SiNP composite materials or DNA-SiNP-CNT composite materials with varying proportions of silica nanoparticles of 0-100% and carbon nanotubes of 0-100%. To link the materials, for example chlorohexyl-modified nucleic acids are incorporated into the DNA polymer by a polymerase reaction and are bonded to the protein material via a self-ligating protein, e.g., the so-called HOB protein (halo-based oligonucleotide binder protein) (K. J. Koßmann et al., Chembiochem. 2016, 17, 12, 1102-1106).

In order to build catalytically active DNA-protein hybrid materials, in the method according to the invention, the catalytically active proteins (6) are genetically fused both to the connectors (7) or (8) and to the HOB protein in step A). Thus, the catalytically active proteins (6) can be coupled to one another both via the complementary connectors (7) and (8) and simultaneously coupled via the HOB protein, for example to a DNA-SiNP composite material.

In the method according to embodiments of the invention, foaming the catalytically active proteins has the surprising effect that the catalytically active proteins which are coupled to one another do not denature during foaming, as is generally observed for enzymes under mechanical stresses by shear forces.

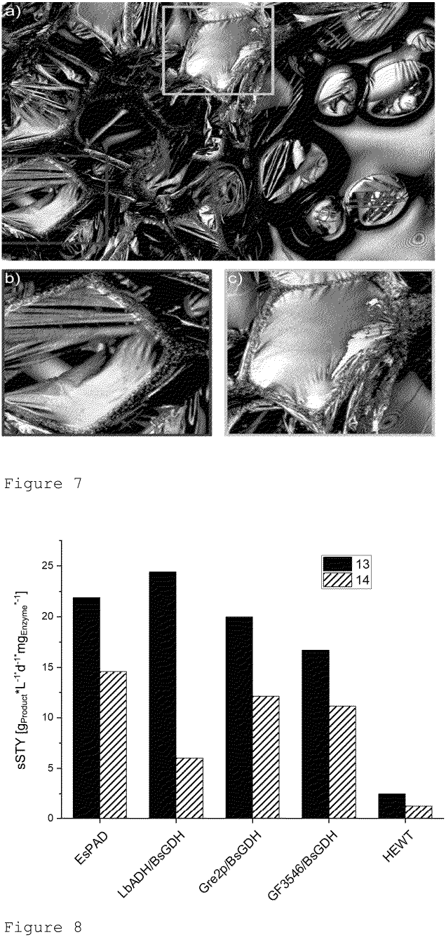

Moreover, the connectors (7) and (8) used lead to a stable connection of the proteins in the form of a foam in which the bubbles no longer coalesce. Surprisingly, in the protein foam (10) according to embodiments of the invention, sensitive proteins, such as the enzyme Saccharomyces cerevisiae NADP(H)-dependent alcohol dehydrogenase (Gre2P) can also be used, although this enzyme rapidly develops and loses its activity already in homogeneous solution with vigorous stirring. Also for further catalytically active proteins (6), such as Enterobacter sp. phenacrylate decarboxylase (EsPAD), Lactobacillus brevis alcohol dehydrogenase (LbADH), Bacillus subtilis glucose 1-dehydrogenase (BsGDH), Streptomnyces sp. (S)-imine reductase (GF3546) and Halomonas elongata amine transaminase (HEWT), it was possible to shown that the catalytic activity in the form of the corresponding catalytically active protein foams (10) is maintained and the catalytic productivity is increased compared to the corresponding monolithic enzyme materials (14) (“all-enzyme hydrogels”, see T. Peschke et al., Angew. Chem. Int. Ed. 2018, 57, 17028-17032) (FIG. 8).

In addition, the catalytically active protein foams (10) obtained with the method according to embodiments of the invention show an unexpected aging effect which increases the catalytic activity of these foams. Whereas, in the foam formation process, the bubbles initially arrange themselves next to one another and form a plurality of bubble layers (9), a “hardening” of the protein foams can be observed in the further course of time, in which the proteins cross-link in a defined manner via connectors at the molecular level. During this maturation of the catalytically active protein foams, the mechanical strength thereof is also increased.

Compared to the “all-enzyme hydrogels” known from the prior art (T. Peschke et al., Angew. Chem. Int. Ed. 2018, 57, 17028-17032), the protein foams (10) produced according to the method according to embodiments of the invention have a significantly larger surface area, which is advantageous for use as immobilized catalysts, for example.

FIG. 1 shows the schematic representation of a bubble generator for producing bubbles containing catalytically active proteins (6). Via the inlet (1), the solution of catalytically active proteins fused to connectors (continuous phase) is introduced into the system, and a gas is introduced as the disperse phase via the inlet (2). Both phases meet at the intersection (3) on which the gas phase is separated into individual bubbles by the continuous phase. These flow through the outlet (4) from the structure into a reaction chamber (5) (FIG. 2).

FIG. 2 shows the schematic representation of the reaction chamber (5) for the flow-catalytic processing of the protein foam. FIG. 2a) shows an isometric view of the reaction chamber. FIG. 2b) shows the side view and FIG. 2c) shows the top view of the structure. In order to fluidically contact the structure, it tapers over a length of 4 mm from the channel width of 3 mm to 1 mm to the ends. The protein foam is located in the 1 mm high, 3 mm wide and 54 mm long reaction chamber in the dried state in a tightly packed sphere packing.

FIG. 3 schematically shows the entire process of producing a catalytically active protein foam (10): First, catalytically active proteins (6) connected to a connector (7) or a connector (8) are introduced as a solution into the bubble generator (FIG. 1). The bubbles generated in the bubble generator are transferred via the outlet channel (4) into the reaction chamber (5). The individual bubbles which are separated by a liquid phase which contains the catalytically active proteins fused to the connectors (7) or (8) are shown schematically in the reaction chamber (5). Individual molecules of the catalytically active proteins are coupled to one another via the mutually complementary connectors (7) and (8). The bubbles are initially arranged next to one another and form a plurality of bubble layers (9). In the protein foam (10) then formed, proteins combine through the connectors (7) and (8) in a “maturation process”, i.e., properties such as the mechanical strength change over time. The resulting material can be used to convert for example a substrate (11) to a product (12).

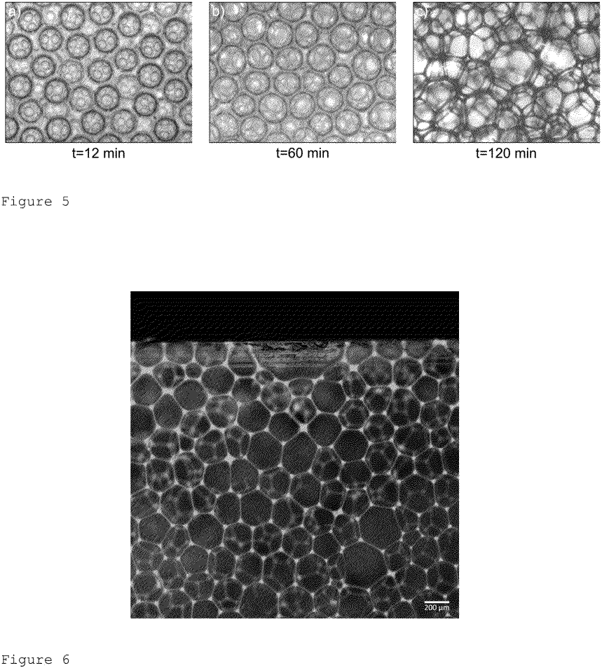

FIG. 4 shows high-speed recordings of the reproducible generation of monodisperse bubbles, which were produced by means of the bubble generator from FIG. 1, and their spatial arrangement in the outlet channel (4), depending on the pressures applied. The pressure of the continuous phase was kept constant at p = 330 mbar. The pressure of the disperse phase corresponds to a) 80 mbar, b) 90 mbar, c) 100 mbar, d) 110 mbar, e) 130 mbar, f) 140 mbar, g) 150 mbar.

At a pressure of approximately 110 mbar and higher, the bubbles begin to be arranged next to one another. As a result of the increase in pressure of the disperse phase, an increase in the bubble diameter can be seen at the same time.

In FIG. 5, the time-dependent cross-linking of the protein foam is documented by transmitted light microscopy. Initially, monodisperse foam cells can be seen which partially coalesce over a period of 2 hours under room temperature at constant evaporation of the aqueous component and form a stable matrix. FIG. 5a) shows the protein foam in the reactor chamber after 12 minutes. The bubbles arrange themselves as hexagonally densely packed Kepler FCC (face centered cubic) sphere packings. FIG. 5b) shows an intermediate drying step after 60 minutes. After drying and thus reduction of the liquid fraction, the sphere packing transitions into an arrangement after about 2 hours which is similar to a Kelvin BCC (body-centered cubic) packing (FIG. 5c)).

FIG. 6 shows a fluorescence microscopic image of the interfaces of the dried protein foam matrix. Prior to cross-linking, the proteins were mixed with a fluorescent dye (fluorescein-5-isothiocyanate) to make the interfaces consisting of cross-linked catalytically active proteins visible.

FIG. 7 shows an image of the catalytically active protein foam (10) with a confocal laser scanning microscope. On the top layer of the protein foams, intact thin films have formed, inter alia. Dark regions show the lamellae between the bubbles, light regions the outer side of the bubbles covered with a cross-linked protein thin film (FIG. 7a)). FIG. 7b) shows a detail of a bubble in which this thin film is perforated due to external forces. FIG. 7c) shows a closed, intact thin film.

FIG. 8 illustrates the reactor performance of various, catalytically active protein foam systems. For this purpose, the catalytic productivity of the catalytically active protein foams (10) was tested using the example of enzyme foams (13) under flow conditions in a microreactor and was compared with already published monolithic enzyme materials (14) (“all-enzyme hydrogels”, see T. Peschke et al., Angew. Chem. Int. Ed. 2018, 57, 17028-17032) (Example 6). To calculate the specific space-time yields (sSTY), the respective substrate conversion was evaluated after one hour under continuous operation of the reactor. It was possible to show that the specific space-time yields of the foam materials (enzyme foams (13)) exceed the sSTY of the tightly packed monolithic enzyme materials (14) up to 4 times. The tested catalytically active proteins are selected from Enterobacter sp. phenacrylate decarboxylase (EsPAD), Lactobacillus brevis alcohol dehydrogenase (LbADH), Bacillus subtilis glucose 1-dehydrogenase (BsGDH), Saccharomyces cerevisiae NADP(H)-dependent alcohol dehydrogenase (Gre2P), Streptomnyces sp. (S)-imine reductase (GF3546), Halomonas elongata amine transaminase (HEWT).

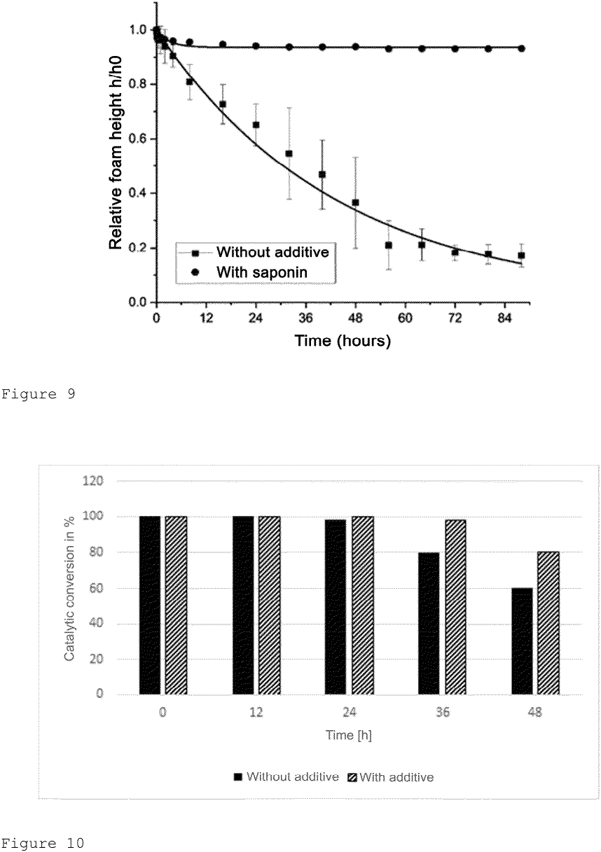

FIG. 9 shows the stability of the catalytically active protein foam (10) on the basis of the relative foam height in a cuvette with and without addition of additives over a period of > 84 h. 35.4 µM saponin was used as the additive.

FIG. 10 shows the catalytic conversion of the catalytically active protein foam (10) after 48 h. Here, the protein foams (10) stabilized by means of surfactant have a 20% higher catalytic conversion than the non-stabilized protein foams (10).

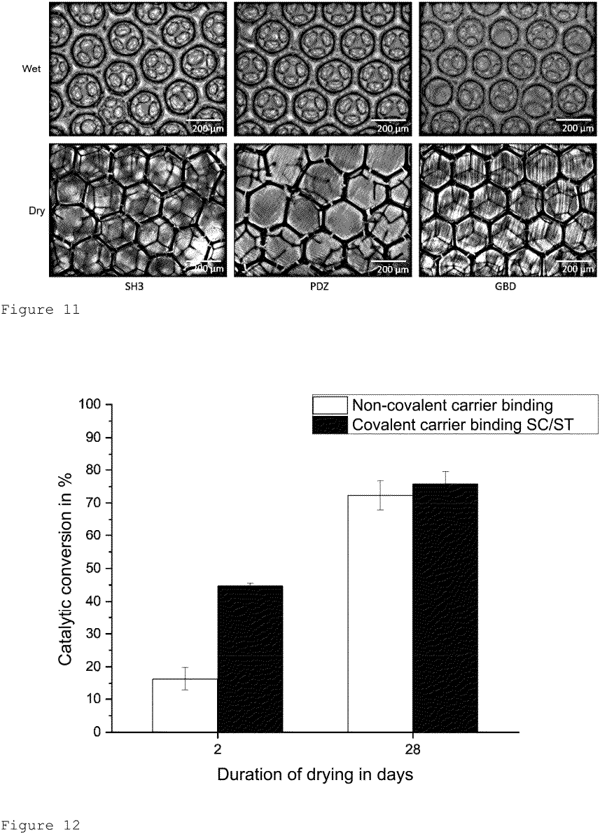

FIG. 11 shows examples of the production of the catalytically active protein foams (10) which consist of the enzymes LbADH/BsGDH, which are genetically fused to the non-covalent protein/protein interaction domains SH3, PDZ and GBD and their corresponding ligands. The time-dependent cross-linking of the protein foam is documented by transmitted light microscopy. Initially, monodisperse wet, i.e., non-dried foam cells can be seen (upper image series) which are dried over a period of 30 minutes at 30° C. in an incubator under constant air convection and form a stable uniformly cross-linked matrix (lower image series).

FIG. 12 shows the catalytic conversion at the time t =30 h of flow bioreactors as a function of the drying duration using the enzyme system LbADH/BsGDH, which is linked either by means of the covalent connector pair SC/ST or by means of the non-covalent connector pair of the protein/protein interaction domain SH3 and the corresponding ligand. Whereas, in the case of a drying duration of two days, both the covalent and the non-covalent linking of the enzymes do not generate optimal conversions, prolonged drying for 28 days results in a significant, approximately 4-fold improvement in the catalytic conversion.

The catalytically active protein foams of the method according to embodiments of the invention can be used in the field of biotechnology, biocatalysis and microfluidics. The protein foams are suitable in particular as carrier-free, immobilized material for flow biocatalysis, for example in flow bioreactors for continuous production processes.

### EXAMPLES

### Example 1

### Bubble Generator Structure and Function

The bubble generator used in the method according to embodiments of the invention (FIG. 1) has as a first element an inlet for the continuous phase (1), which is divided into two mirror-symmetrical paths. These impinge on the inlet channel of the disperse phase (2) at a 45° angle from both sides. In the example shown here, the channel cross-section is 400 µm × 100 µm over a length of 31.77 mm and tapers to form a cross-section of 100 µm × 100 µm over a length of 3.59 mm shortly before the two channels are joined. The channel of the disperse phase intersects the channel cross-section of the continuous phase in its mirror axis and thus generates the functional element of the flow-focusing intersection (3). In the example shown here, the intersection has a cross-section of 100 µm × 100 µm and a length of 31.828 mm. The fourth channel represents the outlet channel (4) for the bubbles generated at the intersection. In the example shown here, it has the dimensions 350 µm × 200 µm × 20.23 mm (WxHxL). The perpendicular inlet and outlet channels have a diameter of 800 µm. The inlets and outlets of the bubble generator are connected via special µchip-to-world (µCTW) connectors. The seals between the chip and the interface are secured by O-rings from NBR 70. On the opposite side, the µCTW connector has connections for standardized fittings and hoses, which are in turn connected to the precision pressure regulators.

As phases for the bubble generation, nitrogen was used as the gas phase and an aqueous solution of catalytically active proteins that are fused to the connectors (7) or (8) was used as the continuous phase. The concentration of the protein solution was between 1 µM and 100 mM depending on the desired polymerization time. A concentration of 1 mM of the protein solution proved to be well suited. The speeds of the continuous and disperse phase were regulated via a pressure gradient Δp. In this example, a pressure gradient of Δp = 330 mbar was experimentally determined for the bubble size shown here. For bubble generation, the optimum pressure gradient for the disperse phase was Δp = 150 mbar.

### Example 2

### Generation of a Microfluidic Catalytically Active Protein Foam

The catalytically active protein foam was produced using the bubble generator described in Example 1 and FIG. 1. To this end, an aqueous solution of catalytically active proteins fused to connectors (7) and an aqueous solution of catalytically active proteins fused to complementary connectors (8) were mixed with one another in a stoichiometric ratio and incubated for 30 minutes at room temperature. The solution was then connected to the bubble generator. The bubble generator was first flooded with N₂ gas and constantly flowed through. Subsequently, the protein solution was introduced into the microfluidic unit at a constant flow rate and foamed at the intersection nozzle (intersection (3)) with the aid of the N₂ gas. The foamed material was collected at the outlet and 100 µL were directly transferred into a prepared polydimethylsiloxane reaction chamber (5).

### Example 3

### Production of Catalytically Active Proteins With Genetically Fused SpyCatcher or SpyTag Domain (Table 1) for Polymerization

For the heterologous expression of 6 catalytically active proteins fused to SpyCatcher or SpyTag, selected from Enterobacter sp. phenacrylate decarboxylase (EsPAD), Lactobacillus brevis alcohol dehydrogenase (LbADH), Bacillus subtilis glucose 1-dehydro-genase (BsGDH), Saccharomyces cerevisiae NADP(H)-dependent alcohol dehydrogenase (Gre2P), Streptomnyces sp. (S)-imine reductase (GF3546), and Halomonas elongata amine transaminase (HEWT), E. coli BL21 (DE3) was transformed with the corresponding expression vector by means of heat shock transformation. The freshly transformed E. coli cells which carry the protein-encoding plasmids were selected overnight on LB agar plates with 100 µg/ml ampicillin at 37° C. Liquid cultures of 160 ml LB medium mixed with ampicillin were produced from clones of the LB agar plates and cultivated overnight for 14-18 hours at 37° C. and 180 rpm in a 500 ml shaking flask. 2 l of ampicillin-containing LB medium was inoculated 1:20 with an overnight culture. The cultures were cultured at 37° C., 180 rpm up to an OD600 of 0.6. The temperature was then lowered to 25° C., IPTG was added to a final concentration of 0.1 mM and the cultures were incubated for a further 16 hours. The cultures were pooled and the cells harvested by centrifugation (10000xg, 10 min) and resuspended in 60 ml of buffer A (50 mM NaH₂PO₄, 300 mM NaCl, 10 mM imidazole, pH 8.0). After digestion through ultrasound, the cell lysate was obtained after centrifugation (45000xg, 1 h), filtered through a 0.45 µm Durapore PVDF membrane (Steriflip, millipore) and charged onto two HisTrap FF (5 ml) Ni-NTA columns (GE Healthcare, Germany) connected in series, which were mounted on an Akta Pure liquid chromatography system (GE Healthcare, Germany). The column was washed with 100 ml of buffer A and the 6xHis-tagged proteins were eluted with a gradient of 100% buffer A to 100% buffer B (50 mM NaH₂PO₄, 300 mM NaCl, 500 mM imidazole, pH 8.0) over a volume of 200 ml. Subsequently, the buffer with Vivaspin 10000 MWCO (GE Healthcare) was replaced by a suitable storage buffer. The genetic constructions were carried out with the isothermal recombination (D. G. Gibson et al., Nat Methods. 2009, 6, 5, 343-345) using oligonucleotide primers with 30 bp (base pairs) homologous overlaps. After assembly, the reaction mixtures were treated with the restriction enzyme DpnI to remove all remaining vectors from previous PCR reactions, and were then transformed into E. coli DH5α cells. All plasmids were cleaned with ZR Plasmid Miniprep-Classic (Zymo Research, Germany) according to the manufacturer’s instructions, and the sequence was verified by commercial sequencing (LGC genomics, Germany).

Table 1: DNA sequences for the heterologous expression of catalytically active proteins fused with SpyCatcher or SpyTag or with the non-covalent protein/protein interaction domains SH3, PDZ and GBD and their corresponding ligands (SC = SpyCatcher domain; ST or ST2 = SpyTag domains; SH3, PDZ or GBD-BD = SH3, PDZ or GBD-bond domain; SH3, PDZ or GBD-L = SH3, PDZ or GBD-ligand.

| Name | Sequence |
|---|---|
| EsPAD-ST2 (SEQ ID NO: 1) | ATGGCTCACATCGTTATGGTGGACGCTTACAAACCGACCAAA GGTGGTGGTGGTAGCAACACCTTCGACAAACATGATCTGAGC GGTTTTGTTGGTAAACATCTGGTGTATACCTATGATAATGGCT GGGAGTATGAGATCTATGTGAAAAATGAAAACACCCTGGATT ATCGCATTCATAGCGGTCTGGTTGGTAATCGTTGGGTTAAAGA TCAGCAGGCATATATTGTTCGTGTGGGTGAAAGCATCTATAA AATCAGCTGGACCGAACCGACCGGCACCGATGTTAGCCTGAT TGTTAATCTGGGTGATAGCCTGTTTCATGGCACCATCTTTTTTC CGCGTTGGGTGATGAATAATCCGGAAAAAACCGTTTGCTTTC AGAACGATCATATTCCGCTGATGAATAGCTATCGTGATGCAG GTCCGGCATATCCGACCGAAGTTATTGATGAATTTGCCACCAT |
| | TACCTTTGTTCGTGATTGTGGTGCAAATAACGAAAGCGTTATT GCATGTGCAGCAAGCGAACTGCCGAAAAACTTTCCGGATAAT CTGAAAGGTGGTGGTGGTAGCGCACATATTGTTATGGTTGAT GCATATAAACCGACCAAAGGTCATCATCACCATCATCATTAA |
| EsPAD-SC (SEQ ID NO: 2) | ATGAACACCTTCGACAAACATGATCTGAGCGGTTTTGTTGGTA AACATCTGGTGTATACCTATGATAATGGCTGGGAGTATGAGA TCTATGTGAAAAATGAAAACACCCTGGATTATCGCATTCATA GCGGTCTGGTTGGTAATCGTTGGGTTAAAGATCAGCAGGCAT ATATTGTTCGTGTGGGTGAAAGCATCTATAAAATCAGCTGGA CCGAACCGACCGGCACCGATGTTAGCCTGATTGTTAATCTGG GTGATAGCCTGTTTCATGGCACCATCTTTTTTCCGCGTTGGGT GATGAATAATCCGGAAAAAACCGTTTGCTTTCAGAACGATCA TATTCCGCTGATGAATAGCTATCGTGATGCAGGTCCGGCATAT CCGACCGAAGTTATTGATGAATTTGCCACCATTACCTTTGTTC GTGATTGTGGTGCAAATAACGAAAGCGTTATTGCATGTGCAG CAAGCGAACTGCCGAAAAACTTTCCGGATAATCTGAAAGGTG GTGGTGGTAGCGTTGATACCCTGAGCGGTCTGAGCAGCGAAC AGGGTCAGAGCGGTGATATGACCATTGAAGAAGATAGCGCAA CCCACATCAAATTCAGCAAACGTGATGAAGATGGTAAAGAAC TGGCAGGCGCAACCATGGAACTGCGTGATAGCAGCGGTAAAA CCATTAGCACCTGGATTAGTGATGGTCAGGTGAAAGATTTTTA TCTGTACCCTGGCAAATACACCTTTGTTGAAACCGCAGCACCG GATGGTTATGAAGTTGCAACCGCAATTACCTTTACCGTTAATG AACAGGGCCAGGTTACCGTGAATGGTAAAGCAACCAAAGGTG ATGCACATATTGGTCATCATCACCATCATCATTAA |
| LbADH-SC (SEQ ID NO: 3) | ATGCATCATCACCACCATCATGGTGTTGATACCCTGAGCGGTC TGAGCAGCGAACAGGGTCAGAGCGGTGATATGACCATTGAAG AAGATAGCGCAACCCACATCAAATTCAGCAAACGTGATGAAG ATGGTAAAGAACTGGCAGGCGCAACCATGGAACTGCGTGATA GCAGCGGTAAAACCATTAGCACCTGGATTAGTGATGGTCAGG TGAAAGATTTTTATCTGTACCCTGGCAAATACACCTTTGTTGA AACCGCAGCACCGGATGGTTATGAAGTTGCAACCGCAATTAC CTTTACCGTTAATGAACAGGGCCAGGTTACCGTGAATGGTAA |
| | AGCAACCAAAGGTGATGCACATATTGGTGGTGGTGGTAGCAG CAATCGCCTGGATGGTAAAGTTGCAATTATTACCGGTGGCAC CCTGGGTATTGGTCTGGCAATTGCAACCAAATTTGTTGAAGAG GGTGCCAAAGTTATGATTACCGGTCGTCATAGTGATGTTGGTG AAAAAGCAGCAAAAAGCGTTGGTACACCGGATCAGATTCAGT TTTTTCAGCATGATAGCAGTGATGAAGATGGTTGGACCAAAC TGTTTGATGCAACCGAAAAAGCATTTGGTCCGGTTAGCACCCT GGTTAATAATGCAGGTATTGCAGTGAATAAAAGCGTGGAAGA AACCACCACCGCAGAATGGCGTAAACTGCTGGCAGTTAATCT GGATGGTGTTTTTTTTGGTACACGTCTGGGTATTCAGCGCATG AAAAACAAAGGTCTGGGTGCAAGCATTATCAACATGAGCAGC ATTGAAGGTTTTGTTGGTGATCCGAGCCTGGGTGCATATAATG CAAGCAAAGGTGCAGTTCGTATTATGAGCAAAAGCGCAGCAC TGGATTGTGCACTGAAAGATTATGATGTTCGTGTGAATACCGT TCATCCGGGTTATATCAAAACACCGCTGGTTGATGATCTGCCT GGTGCCGAAGAAGCAATGAGCCAGCGTACCAAAACCCCGATG GGTCATATTGGTGAACCGAATGATATTGCCTATATCTGTGTTT ATCTGGCCAGCAACGAAAGTAAATTTGCAACCGGTAGCGAAT TTGTTGTGGATGGTGGTTATACCGCACAGTAA |
| BsGDH-ST (SEQ ID NO: 4) | ATGTATCCGGATTTAAAAGGAAAAGTCGTCGCTATTACAGGA GCTGCTTCAGGGCTCGGAAAGGCAATGGCCATTCGCTTCGGC AAGGAGCAGGCAAAAGTGGTTATCAACTATTATAGTAATAAA CAAGATCCGAACGAGGTAAAAGAAGAGGTCATCAAGGCGGG CGGTGAAGCTGTTGTCGTCCAAGGAGATGTCACGAAAGAGGA AGATGTAAAAAATATCGTGCAAACGGCAATTAAAGAGTTCGG CACACTCGATATTATGATTAATAATGCCGGTCTTGAAAATCCT GTGCCATCTCACGAAATGCCGCTTAAGGATTGGGATAAAGTC ATCGGCACAAACTTAACAGGTGCCTTTTTAGGAAGCCGTGAA GCGATTAAATATTTCGTAGAAAACGATATCAAGGGAAATGTC ATCAACATGTCCAGTGTGCACGAAGTGATTCCTTGGCCGTTAT TTGTTCACTACGCGGCAAGTAAAGGCGGGATAAAGCTGATGA CAGAAACATTGGCGTTGGAATACGCGCCGAAGGGCATTCGCG TGAACAATATCGGGCCAGGTGCGATCAATACGCCAATCAATG |
| | CTGAAAAATTTGCTGACCCTAAACAGAAAGCAGATGTAGAAA GCATGATTCCGATGGGGTATATCGGCGAACCGGAGGAGATCG CCGCAGTGGCAGTGTGGCTTGCTTCGAAGGAATCCAGCTATG TTACAGGCATCACATTGTTTGCGGACGGCGGAATGACGAAAT ATCCTTCTTTCCAGGCAGGACGCGGTGGTGGTGGTGGTAGCG GTGGTGGTGGTAGCGCCCATATTGTTATGGTGGATGCATATAA ACCGACCAAAGGTCATCATCACCATCACCATTGA |
| Gre2p-ST2 (SEQ ID NO: 5) | ATGGCTCACATCGTTATGGTTGACGCTTACAAACCGACCAAA GGTGGTGGTGGTAGCTCAGTTTTTGTTTCAGGTGCTAACGGGT TCATTGCCCAACACATTGTCGATCTCCTATTGAAGGAAGACTA TAAGGTCATCGGTTCTGCTAGAAGTCAAGAAAAGGCCGAGAA TTTAACGGAGGCCTTTGGTAACAATCCAAACTTCTCCATGGAA ATTGTCCCAGACATATCTAAGCTGGACGCATTTGACCATGTTT TCCAAAAGCACGGCAAGGAGATCAAGATAGTTCTACATACGG CCTCTCCATTCTGCTTTGATATCACTGACAGTGAACGCGATTT ATTAATTCCTGCTGTGAACGGTGTTAAGGGAATTCTCCACTCA ATTAAAAAATACGCCGCTGATTCTGTAGAACGTGTAGTTCTCA CCTCTTCTTATGCAGCTGTGTTCGACATGGCAAAAGAAAACG ATAAATCTTTAACATTTAACGAAGAATCCTGGAACCCAGCAA CCTGGGAGAGTTGCCAAAGTGACCCGGTTAGCGCCTACTGTG GTTCTAAGAAGTTTGCTGAAAAAGCAGCTTGGGAATTTCTAG AGGAGAATAGAGACGCTGTAAAATTCGAATTAACTGCCGTTA ACCCAGTTTACGTTTTTGGTCCGCAAATGTTTGACAAAGATGT GAAAAAACACTTGAACACATCTTGCGAACTCGTCAACAGCTT GATGCATTTATCACCAGAGGACAAGATACCGGAACTATTTGG TGGATACATTGATGTTCGTGATGTTGCAAAGGCTCATTTAGTT GCCTTCCAAAAGAGGGAAACAATTGGTCAAAGACTAATCGTA TCAGAGGCCAGATTTACTATGCAGGATGTTCTCGATATCCTTA ACGAAGACTTCCCTATCCTAAAAGGCAATATTCCAGTGGGGA AACCAGGTTCTGGTGCTACCCATAACACCCTTGGTGCTACTCT TGATAATAAAAAGAGTAAGAAATTGTTAGGTTTCAAGTTCAG GAACTTGAAAGAGACCATTGACGACACTGCCTCCCAAATTTT AAAATTTGAGGGCAGAATAGGTGGTGGTGGTAGCGCCCATAT |
| | TGTTATGGTGGATGCATATAAACCGACCAAAGGTCATCATCA CCATCACCATTGAGTTTGA |
| BsGDH-SC (SEQ ID NO: 6) | ATGTATCCGGATTTAAAAGGAAAAGTCGTCGCTATTACAGGA GCTGCTTCAGGGCTCGGAAAGGCAATGGCCATTCGCTTCGGC AAGGAGCAGGCAAAAGTGGTTATCAACTATTATAGTAATAAA CAAGATCCGAACGAGGTAAAAGAAGAGGTCATCAAGGCGGG CGGTGAAGCTGTTGTCGTCCAAGGAGATGTCACGAAAGAGGA AGATGTAAAAAATATCGTGCAAACGGCAATTAAAGAGTTCGG CACACTCGATATTATGATTAATAATGCCGGTCTTGAAAATCCT GTGCCATCTCACGAAATGCCGCTTAAGGATTGGGATAAAGTC ATCGGCACAAACTTAACAGGTGCCTTTTTAGGAAGCCGTGAA GCGATTAAATATTTCGTAGAAAACGATATCAAGGGAAATGTC ATCAACATGTCCAGTGTGCACGAAGTGATTCCTTGGCCGTTAT TTGTTCACTACGCGGCAAGTAAAGGCGGGATAAAGCTGATGA CAGAAACATTGGCGTTGGAATACGCGCCGAAGGGCATTCGCG TGAACAATATCGGGCCAGGTGCGATCAATACGCCAATCAATG CTGAAAAATTTGCTGACCCTAAACAGAAAGCAGATGTAGAAA GCATGATTCCGATGGGGTATATCGGCGAACCGGAGGAGATCG CCGCAGTGGCAGTGTGGCTTGCTTCGAAGGAATCCAGCTATG TTACAGGCATCACATTGTTTGCGGACGGCGGAATGACGAAAT ATCCTTCTTTCCAGGCAGGACGCGGTGGTGGTGGTGGTAGCG GTGGTGGTGGTAGCGTTGATACCCTGAGCGGTCTGAGCAGCG AACAGGGTCAGAGCGGTGATATGACCATTGAAGAAGATAGCG CAACCCACATCAAATTCAGCAAACGTGATGAAGATGGTAAAG AACTGGCAGGCGCAACCATGGAACTGCGTGATAGCAGCGGTA AAACCATTAGCACCTGGATTAGTGATGGTCAGGTGAAAGATT TTTATCTGTACCCTGGCAAATACACCTTTGTTGAAACCGCAGC ACCGGATGGTTATGAAGTTGCAACCGCAATTACCTTTACCGTT AATGAACAGGGCCAGGTTACCGTGAATGGTAAAGCAACCAAA GGTGATGCACATATTGGTCATCATCACCATCACCATTGA |
| GF3546-ST (SEQ ID NO: 7) | ATGCATCACCACCATCATCATGGTGCCCATATTGTTATGGTTG ATGCCTATAAACCGACCAAAGGTGGTGGTGGTAGCAGCAAAC AGAGCGTTACCGTTATTGGTCTGGGTCCGATGGGTCAAGCAA |
| | TGGTTAATACCTTTCTGGATAATGGTCATGAAGTGACCGTTTG GAATCGTACCGCAAGCAAAGCCGAAGCACTGGTTGCACGTGG TGCAGTTCTGGCACCGACCGTTGAAGATGCACTGAGCGCAAA TGAACTGATTGTTCTGAGCCTGACCGATTATGATGCAGTTTAT GCAATTCTGGAACCGGTTACCGGTAGCCTGAGCGGTAAAGTT ATTGCAAATCTGAGCAGCGATACACCGGATAAAGCACGTGAA GCAGCAAAATGGGCAGCAAAACATGGTGCAAAACATCTGACC GGTGGTGTTCAGGTTCCGCCTCCGCTGATTGGTAAACCGGAA AGCAGCACCTATTATAGCGGTCCGAAAGATGTTTTTGATGCCC ATGAAGATACCCTGAAAGTTCTGACCAATGCAGATTATCGTG GTGAAGATGCCGGTCTGGCAGCAATGTATTATCAGGCACAGA TGACCATTTTTTGGACCACCATGCTGAGCTATTATCAGACCCT GGCATTAGGTCAGGCAAATGGTGTTAGCGCCAAAGAACTGCT GCCGTATGCAACAATGATGACCAGCATGATGCCGCATTTTCTG GAACTGTATGCACAGCATGTTGATAGTGCCGATTATCCGGGT GATGTTGATCGTCTGGCAATGGGTGCAGCAAGCGTTGATCAT GTTCTGCATACCCATCAGGATGCCGGTGTTAGCACCGTTCTGC CTGCAGCAGTTGCAGAAATCTTTAAAGCAGGTATGGAAAAAG GCTTTGCCGAAAATAGCTTTAGCAGCCTGATTGAGGTTCTGAA AAAACCGGCAGTTTAA |
| HEWT-ST2 (SEQ ID NO: 8) | ATGCATCACCACCATCATCATGGTGCCCATATTGTTATGGTTG ATGCCTATAAACCGACCAAAGGTGGTGGTGGTAGTCAGACCC AGGATTATCAGGCACTGGATCGTGCACATCATCTGCATCCGTT TACCGATTTTAAAGCACTGGGTGAAGAAGGTAGTCGCGTTGT TACCCATGCCGAAGGTGTTTATATCCATGATAGCGAAGGTAA TCGTATTCTGGATGGTATGGCAGGTCTGTGGTGTGTTAATTTA GGTTATGGTCGTCGTGAACTGGTTGAAGCAGCAACCGCACAG CTGGAACAGCTGCCGTATTATAACACCTTTTTCAAAACCACAC ATCCGCCTGCAGTTCGTCTGGCAGAAAAACTGTGTGATCTGGC ACCGGCACATATTAATCGTGTGTTTTTTACCGGTAGCGGTAGC GAAGCAAATGATACCGTTCTGCGTATGGTTCGTCGTTATTGGG CACTGAAAGGTCAGCCGGATAAACAGTGGATTATTGGTCGTG AAAATGCCTATCATGGTAGCACCCTGGCAGGTATGAGCTTAG |
| | GTGGCATGGCACCGATGCATGCACAAGGTGGTCCGTGTGTTC CGGGTATTGCACATATTCGTCAGCCGTATTGGTTTGGTGAAGG TCGTGATATGAGTCCGGAAGCATTTGGTCAGACCTGTGCAGA AGCACTGGAAGAAAAAATTCTGGAACTGGGCGAAGAAAAAG TGGCAGCATTTATTGCAGAACCGGTGCAAGGTGCCGGTGGTG CAATTATGCCTCCGGAAAGCTATTGGCCTGCCGTTAAAAAAG TTCTGGCCAAATATGATATTCTGCTGGTTGCCGATGAAGTGAT TTGCGGTTTTGGTCGTTTAGGTGAATGGTTTGGTAGCCAGCAT TATGGTCTGGAACCGGATCTGATGCCGATTGCAAAAGGTCTG AGCAGCGGTTATCTGCCGATTGGTGGTGTTCTGGTTGGTGATC GTGTTGCAGAAACCCTGATCGAAGAAGGTGGCGAATTTTTCC ATGGTTTTACCTATAGCGGTCATCCGACCTGTGCCGCAGTTGC ACTGAAAAACCTGGAACTGCTGGAAGCGGAAGGTGTTGTGGA TCGTGTTCGTGATGATCTGGGTCCGTATCTGGCCGAACGTTGG GCAAGCCTGGTTGATCATCCGATTGTTGGTGAAGCACGTAGC CTGGGCCTGATGGGTGCACTGGAATTAGTTGCAGATAAAACC ACCGGTCAGCGTTTTGATAAAAGTTTAGGTGCAGGTAATCTGT GTCGTGATCTGTGTTTTGCCAATGGTCTGGTTATGCGTAGCGT TGGCGATACCATGATTATTAGCCCTCCGCTGGTTATTCGTCGC GAAGAAATTGATGAACTGGTGGAACTGGCACGTCGTGCCCTG GATGAAACCGCACGCCAGCTGACCCAGGTTCCGCATACACAA GAAGAACCGACCGCAGGTGGTGGTGGTAGTGCCCATATTGTT ATGGTTGATGCCTATAAACCGACCAAATAA |
| HEWT-SC (SEQ ID NO: 9) | ATGCATCATCACCACCATCATGGTGTTGATACCCTGAGCGGTC TGAGCAGCGAACAGGGTCAGAGCGGTGATATGACCATTGAAG AAGATAGCGCAACCCACATCAAATTCAGCAAACGTGATGAAG ATGGTAAAGAACTGGCAGGCGCAACCATGGAACTGCGTGATA GCAGCGGTAAAACCATTAGCACCTGGATTAGTGATGGTCAGG TGAAAGATTTTTATCTGTACCCTGGCAAATACACCTTTGTTGA AACCGCAGCACCGGATGGTTATGAAGTTGCAACCGCAATTAC CTTTACCGTTAATGAACAGGGCCAGGTTACCGTGAATGGTAA AGCAACCAAAGGTGATGCACATATTGGTGGTGGTGGTAGTCA GACCCAGGATTATCAGGCACTGGATCGTGCACATCATCTGCA |
| | TCCGTTTACCGATTTTAAAGCACTGGGTGAAGAAGGTAGTCG CGTTGTTACCCATGCCGAAGGTGTTTATATCCATGATAGCGAA GGTAATCGTATTCTGGATGGTATGGCAGGTCTGTGGTGTGTTA ATTTAGGTTATGGTCGTCGTGAACTGGTTGAAGCAGCAACCG CACAGCTGGAACAGCTGCCGTATTATAACACCTTTTTCAAAAC CACACATCCGCCTGCAGTTCGTCTGGCAGAAAAACTGTGTGA TCTGGCACCGGCACATATTAATCGTGTGTTTTTTACCGGTAGC GGTAGCGAAGCAAATGATACCGTTCTGCGTATGGTTCGTCGTT ATTGGGCACTGAAAGGTCAGCCGGATAAACAGTGGATTATTG GTCGTGAAAATGCCTATCATGGTAGCACCCTGGCAGGTATGA GCTTAGGTGGCATGGCACCGATGCATGCACAAGGTGGTCCGT GTGTTCCGGGTATTGCACATATTCGTCAGCCGTATTGGTTTGG TGAAGGTCGTGATATGAGTCCGGAAGCATTTGGTCAGACCTG TGCAGAAGCACTGGAAGAAAAAATTCTGGAACTGGGCGAAG AAAAAGTGGCAGCATTTATTGCAGAACCGGTGCAAGGTGCCG GTGGTGCAATTATGCCTCCGGAAAGCTATTGGCCTGCCGTTAA AAAAGTTCTGGCCAAATATGATATTCTGCTGGTTGCCGATGAA GTGATTTGCGGTTTTGGTCGTTTAGGTGAATGGTTTGGTAGCC AGCATTATGGTCTGGAACCGGATCTGATGCCGATTGCAAAAG GTCTGAGCAGCGGTTATCTGCCGATTGGTGGTGTTCTGGTTGG TGATCGTGTTGCAGAAACCCTGATCGAAGAAGGTGGCGAATT TTTCCATGGTTTTACCTATAGCGGTCATCCGACCTGTGCCGCA GTTGCACTGAAAAACCTGGAACTGCTGGAAGCGGAAGGTGTT GTGGATCGTGTTCGTGATGATCTGGGTCCGTATCTGGCCGAAC GTTGGGCAAGCCTGGTTGATCATCCGATTGTTGGTGAAGCAC GTAGCCTGGGCCTGATGGGTGCACTGGAATTAGTTGCAGATA AAACCACCGGTCAGCGTTTTGATAAAAGTTTAGGTGCAGGTA ATCTGTGTCGTGATCTGTGTTTTGCCAATGGTCTGGTTATGCG TAGCGTTGGCGATACCATGATTATTAGCCCTCCGCTGGTTATT CGTCGCGAAGAAATTGATGAACTGGTGGAACTGGCACGTCGT GCCCTGGATGAAACCGCACGCCAGCTGACCCAGGTTCCGCAT ACACAAGAAGAACCGACCGCATAA |
| LbADH- | ATGCATCATCACCACCATCATGGTGCAGAATATGTTCGTGCCC |
| SH3-BD (SEQ ID NO: 10) | TGTTTGATTTTAATGGCAACGATGAAGAAGATCTGCCGTTCAA AAAAGGTGATATTCTGCGTATTCGTGACAAACCGGAAGAACA GTGGTGGAATGCAGAAGATAGCGAAGGTAAACGTGGTATGAT TCCGGTTCCGTATGTGGAAAAATATGGTGGTGGTGGTAGCAG CAATCGCCTGGATGGTAAAGTTGCAATTATTACCGGTGGCAC CCTGGGTATTGGTCTGGCAATTGCAACCAAATTTGTTGAAGAG GGTGCCAAAGTTATGATTACCGGTCGTCATAGTGATGTTGGTG AAAAAGCAGCAAAAAGCGTTGGTACACCGGATCAGATTCAGT TTTTTCAGCATGATAGCAGTGATGAAGATGGTTGGACCAAAC TGTTTGATGCAACCGAAAAAGCATTTGCTCCGGTTAGCACCCT GGTTAATAATGCAGGTATTGCAGTGAATAAAAGCGTGGAAGA AACCACCACCGCAGAATGGCGTAAACTGCTGGCAGTTAATCT GGATGGTGTTTTTTTTGGTACACGTCTGGGTATTCAGCGCATG AAAAACAAAGGTCTGGGTGCAAGCATTATCAACATGAGCAGC ATTGAAGGTTTTCTTGGTGATCCGAGCCTGGGTGCATATAATG CAAGCAAAGGTGCAGTTCGTATTATGAGCAAAAGCGCAGCAC TGGATTGTGCACTGAAAGATTATGATGTTCGTGTGAATACCGT TCATCCGGGTTATATCAAAACACCGCTGGTTGATGATCTGCCT GGTGCCCAAGAAGCAATGAGCCAGCGTACCAAAACCCCGATG GGTCATATTGGTGAACCGAATGATATTGCCTATATCTGTGTTT ATCTGGCCAGCAACGAAAGTAAATTTGCAACCGGTAGCGAAT TTGTTGTGGATGGTGGTTATACCGCACAGTAA |
| BsGDH-SH3-L (SEQ ID NO: 11) | ATGTATCCGGATTTAAAAGGAAAAGTCGTCGCTATTACAGGA GCTGCTTCAGGGCTCGGAAAGGCAATGGCCATTCGCTTCGGC AAGGAGCAGGCAAAAGTGGTTATCAACTATTATAGTAATAAA CAAGATCCGAACGAGGTAAAAGAAGAGGTCATCAAGGCGGG CGGTGAAGCTGTTGTCGTCCAAGGAGATGTCACGAAAGAGGA AGATGTAAAAAATATCGTGCAAACGGCAATTAAAGAGTTCGG CACACTCGATATTATGATTAATAATGCCGGTCTTGAAAATCCT GTGCCATCTCACGAAATGCCGCTTAAGGATTGGGATAAAGTC ATCGGCACAAACTTAACAGGTGCCTTTTTAGGAAGCCGTGAA GCGATTAAATATTTCGTAGAAAACGATATCAAGGGAAATGTC ATCAACATGTCCAGTGTGCACGAAGTGATTCCTTGGCCGTTAT |
| | TTGTTCACTACGCGGCAAGTAAAGGCGGGATAAAGCTGATGA CAGAAACATTGGCGTTGGAATACGCGCCGAAGGGCATTCGCG TGAACAATATCGGGCCAGGTGCGATCAATACGCCAATCAATG CTGAAAAATTTGCTGACCCTAAACAGAAAGCAGATGTAGAAA GCATGATTCCGATGGGGTATATCGGCGAACCGGAGGAGATCG CCGCAGTGGCAGTGTGGCTTGCTTCGAAGGAATCCAGCTATG TTACAGGCATCACATTGTTTGCGGACGGCGGAATGACGAAAT ATCCTTCTTTCCAGGCAGGACGCGGTGGTGGTGGTGGTAGCG GTGGTGGTGGTAGCCCGCCGCCGGCGCTGCCGCCGAAACGTC GTCGTGGTCATCATCACCATCACCATTGA |
| LbADH-PDZ-BD (SEQ ID NO: 12) | ATGCATCATCACCACCATCATGGTCTGCAGCGTCGTCGTGTTA CCGTTCGTAAAGCAGATGCCGGTGGTCTGGGTATTAGCATTA AAGGTGGTCGTGAAAACAAAATGCCGATCCTGATTAGCAAAA TCTTTAAAGGTCTGGCAGCAGATCAGACCGAAGCACTGTTTGT TGGTGATGCAATTCTGAGCGTTAATGGTGAAGATCTGAGCAG CGCAACCCATGATGAAGCAGTTCAGGCACTGAAAAAAACCGG TAAAGAAGTTGTTCTGGAAGTCAAATACATGAAAGAAGTGAG CCCGTACTTTAAAGGTGGTGGTGGTAGCAGCAATCGCCTGGA TGGTAAAGTTGCAATTATTACCGGTGGCACCCTGGGTATTGGT CTGGCAATTGCAACCAAATTTGTTGAAGAGGGTGCCAAAGTT ATGATTACCGGTCGTCATAGTGATGTTGGTGAAAAAGCAGCA AAAAGCGTTGGTACACCGGATCAGATTCAGTTTTTTCAGCATG ATAGCAGTGATGAAGATGGTTGGACCAAACTGTTTGATGCAA CCGAAAAAGCATTTGGTCCGGTTAGCACCCTGGTTAATAATG CAGGTATTGCAGTGAATAAAAGCGTGGAAGAAACCACCACCC CAGAATGGCGTAAACTGCTGGCAGTTAATCTGGATGGTGTTTT TTTTGGTACACGTCTGGGTATTCAGCGCATGAAAAACAAAGG TCTGGGTGCAAGCATTATCAACATGAGCAGCATTGAAGGTTTT GTTGGTGATCCGAGCCTGGGTGCATATAATGCAAGCAAAGGT GCAGTTCGTATTATGAGCAAAAGCGCAGCACTGGATTGTGCA CTGAAAGATTATGATGTTCGTGTGAATACCGTTCATCCGGGTT ATATCAAAACACCGCTGGTTGATGATCTGCCTGGTGCCGAAG AAGCAATGAGCCAGCGTACCAAAACCCCGATGGGTCATATTG |
| | GTGAACCGAATGATATTGCCTATATCTGTGTTTATCTGGCCAG CAACGAAAGTAAATTTGCAACCGGTAGCGAATTTGTTGTGGA TGGTGGTTATACCGCACAGTAA |
| BsGDH-PDZ-L (SEQ ID NO: 13) | ATGTATCCGGATTTAAAAGGAAAAGTCGTCGCTATTACAGGA GCTGCTTCAGGGCTCGGAAAGGCAATGGCCATTCGCTTCGGC AAGGAGCAGGCAAAAGTGGTTATCAACTATTATAGTAATAAA CAAGATCCGAACGAGGTAAAAGAAGAGGTCATCAAGGCGGG CGGTGAAGCTGTTGTCGTCCAAGGAGATGTCACGAAAGAGGA AGATGTAAAAAATATCGTGCAAACGGCAATTAAAGAGTTCGG CACACTCGATATTATGATTAATAATGCCGGTCTTGAAAATCCT GTGCCATCTCACGAAATGCCGCTTAAGGATTGGGATAAAGTC ATCGGCACAAACTTAACAGGTGCCTTTTTAGGAAGCCGTGAA GCGATTAAATATTTCGTAGAAAACGATATCAAGGGAAATGTC ATCAACATGTCCAGTGTGCACGAAGTGATTCCTTGGCCGTTAT TTGTTCACTACGCGGCAAGTAAAGGCGGGATAAAGCTGATGA CAGAAACATTGGCGTTGGAATACGCGCCGAAGGGCATTCGCG TGAACAATATCGGGCCAGGTGCGATCAATACGCCAATCAATG CTGAAAAATTTGCTGACCCTAAACAGAAAGCAGATGTAGAAA GCATGATTCCGATGGGGTATATCGGCGAACCGGAGGAGATCG CCGCAGTGGCAGTGTGGCTTGCTTCGAAGGAATCCAGCTATG TTACAGGCATCACATTGTTTGCGGACGGCGGAATGACGAAAT ATCCTTCTTTCCAGGCAGGACGCGGTGGTGGTGGTGGTAGCG GTGGTGGTGGTAGCGGTGTTAAAGAATCTCTGGTTGGTCATCA TCACCATCACCATTGA |
| LbADH-GBD-BD (SEQ ID NO: 14) | ATGCATCATCACCACCATCATGGTACCAAAGCAGATATTGGC ACCCCGAGCAATTTTCAGCATATTGGTCATGTTGGTTGGGATC CGAATACCGGTTTTGATCTGAATAATCTGGATCCGGAACTGA AAAACCTGTTTGATATGTGTGGTATTAGCGAAGCACAGCTGA AAGATCGTGAAACCAGCAAAGTGATCTATGACTTTATCGAAA AAACCGGTGGTGTTGAAGCCGTGAAAAATGAACTGCGTCGTC AGGCACCGGGTGGTGGTGGTAGCAGCAATCGCCTGGATCGTA AAGTTGCAATTATTACCGGTGGCACCCTGGGTATTGGTCTGGC AATTGCAACCAAATTTGTTGAAGAGGGTGCCAAAGTTATGAT |
| | TACCGGTCGTCATAGTGATGTTGGTGAAAAAGCAGCAAAAAG CGTTGGTACACCGGATCAGATTCAGTTTTTTCAGCATGATAGC AGTGATGAAGATGGTTGGACCAAACTGTTTGATGCAACCGAA AAAGCATTTGGTCCGGTTAGCACCCTGGTTAATAATGCAGGT ATTGCAGTGAATAAAAGCGTGGAAGAAACCACCACCGCAGA ATGGCGTAAACTGCTGGCAGTTAATCTGGATGGTGTTTTTTTT GGTACACGTCTGGGTATTCAGCGCATGAAAAACAAAGGTCTG GGTGCAAGCATTATCAACATGAGCAGCATTGAAGGTTTTGTT GGTGATCCGAGCCTGGGTGCATATAATGCAAGCAAAGGTGCA GTTCGTATTATGAGCAAAAGCGCAGCACTGGATTGTGCACTG AAAGATTATGATGTTCGTGTGAATACCGTTCATCCGGGTTATA TCAAAACACCGCTGGTTGATGATCTGCCTGGTGCCGAAGAAG CAATGAGCCAGCGTACCAAAACCCCGATGGGTCATATTGGTG AACCGAATGATATTGCCTATATCTGTGTTTATCTGGCCAGCAA CGAAAGTAAATTTGCAACCGGTAGCGAATTTGTTGTGGATGG TGGTTATACCGCACAGTAA |
| BsGHD-GBD-L (SEQ ID NO: 15) | ATGTATCCGGATTTAAAAGGAAAAGTCGTCGCTATTACAGGA GCTGCTTCAGGGCTCGGAAAGGCAATGGCCATTCGCTTCGGC AAGGAGCAGGCAAAAGTGGTTATCAACTATTATAGTAATAAA CAAGATCCGAACGAGGTAAAAGAAGAGGTCATCAAGGCGGG CGGTGAAGCTGTTGTCGTCCAAGGAGATGTCACGAAAGAGGA AGATGTAAAAAATATCGTGCAAACGGCAATTAAAGAGTTCGG CACACTCGATATTATGATTAATAATGCCGGTCTTGAAAATCCT GTGCCATCTCACGAAATGCCGCTTAAGGATTGGGATAAAGTC ATCGGCACAAACTTAACAGGTGCCTTTTTAGGAAGCCGTGAA GCGATTAAATATTTCGTAGAAAACGATATCAAGGGAAATGTC ATCAACATGTCCAGTGTGCACGAAGTGATTCCTTGGCCGTTAT TTGTTCACTACGCGGCAAGTAAAGGCGGGATAAAGCTGATGA CAGAAACATTGGCGTTGGAATACGCGCCGAAGGGCATTCGCG TGAACAATATCGGGCCAGGTGCGATCAATACGCCAATCAATG CTGAAAAATTTGCTGACCCTAAACAGAAAGCAGATGTAGAAA GCATGATTCCGATCGGGTATATCGGCGAACCGGAGGAGATCG CCGCAGTGGCAGTGTGGCTTGCTTCGAAGGAATCCAGCTATG |
| | TTACAGGCATCACATTGTTTGCGGACGGCGGAATGACGAAAT ATCCTTCTTTCCAGGCAGGACGCGGTGGTGGTGGTGGTAGCG GTGGTGGTGGTAGCCTGGTTGGTGCACTGATGCATGTTATGCA GAAACGTAGCCGTGCAATTCATAGCAGTGATGAAGGTGAAGA TCAAGCCGGTGATGAAGATGAGGATGGTCATCATCACCATCA CCATTGA |

### Example 4

In example 4, monodisperse bubbles were produced by means of the bubble generator from FIG. 1 with variation of the pressure of the disperse phase (FIG. 4). The pressure of the continuous phase was kept constant at p = 330 mbar. The pressure of the disperse phase corresponded to a) 80 mbar, b) 90 mbar, c) 100 mbar, d) 110 mbar, e) 130 mbar, f) 140 mbar, g) 150 mbar. At a pressure of approximately 110 mbar and higher, the bubbles began to be arranged next to one another. As a result of the increase in pressure of the disperse phase, an increase in the bubble diameter was simultaneously evident.

### Example 5

In Example 5, the time-dependent cross-linking of the protein foam was demonstrated by transmitted light microscopy. At the beginning of the cross-linking reaction, monodisperse foam cells were discernible in the reaction chamber (5), which partially coalesced over a period of 2 hours under room temperature at constant evaporation of the aqueous component and formed a stable matrix. After 12 minutes of cross-linking time in the reactor chamber, the bubbles arranged as hexagonally tightly packed Kepler FCC (face centered cubic) sphere packings (FIG. 5a). After an intermediate drying step after 60 minutes (FIG. 5b), the sphere packing transitioned after 120 mins. into an arrangement that was similar to a Kelvin BCC (body-centered cubic) packing (FIG. 5c).

### Example 6

In Example 6, the catalytic productivity of the catalytically active protein foam (10) was tested using the example of enzyme foams (13) under flow conditions in a microreactor (FIG. 8). The reactor performance was compared with the reactor performance of already published monolithic enzyme materials (14), the “all-enzyme hydrogels” (T. Peschke et al., Angew. Chem. Int. Ed. 2018, 57, 17028-17032) (FIG. 8). To calculate the specific space-time yields (sSTY), the respective substrate conversion was evaluated after one hour under continuous operation of the reactor. It was possible to show that the specific space-time yields (sSTY) of the foam materials (enzyme foams (13)) exceed those of the tightly packed monolithic enzyme materials (14) by up to 4 times (FIG. 8). The proteins tested are selected from Enterobacter sp. phenacrylate decarboxylase (EsPAD), Lactobacillus brevis alcohol dehydrogenase (LbADH), Bacillus subtilis glucose 1-dehydro-genase (BsGDH), Saccharomyces cerevisiae NADP(H)-dependent alcohol dehydrogenase (Gre2P), Streptomnyces sp. (S)-imine reductase (GF3546), Halomonas elongata amine transaminase (HEWT).

Using the enzyme EsPAD, the substrate p-coumaric acid was converted with a substrate concentration of 5 mM to 4-vinylphenol (E. Mittmann et al., Micromachines 2019, 10, 795). Using the enzyme system LbADH/BsGDH, with regeneration of the cofactor NADPH 5-nitro-nonane-2,8-dione with a concentration of 5 mM was converted to give the corresponding (R)-hydroxy ketones and (R,R)-diol (T. Peschke et al., Angew Chem Int Ed Engl. 2018, 57, 52, 17028-17032). Using the enzyme system Gre2p/BsGDH, with regeneration of the cofactor NADPH, 5-nitro-nonane-2,8-dione with a concentration of 5 mM was converted to the corresponding (S)-hydroxy ketones and (S,S)-diol (P. Bitterwolf P. et al., Chemical Science 2019, 10, 9752). Using the enzyme system GF3546/BsGDH, with regeneration of the cofactor NADPH, 3,4-dihydroisoquinoline with a concentration of 5 mM was converted to 1,2,3,4-tetrahydroisoquinoline (P. Bitterwolf P. et al., Micromachines 2019, 10, 11, 783). Using the enzyme HEWT, 4-nitrobenzaldehyde with a concentration of 1 mM was converted to 4-nitrobenzylamine.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for producing catalytically active protein foams, comprising the steps of
A) Producing catalytically active proteins fused to a first connector and catalytically active proteins fused to a second connector that is complementary to the first connector;
B) Producing bubbles containing catalytically active proteins in a bubble generator using a gas phase and a solution of the catalytically active proteins fused to the first connector and the catalytically active proteins fused to the second connector from step A);
C) Transferring the bubbles produced in step B) into a reaction chamber; and
D) Coupling the catalytically active proteins contained in the bubbles from step B) in the reation chamber to form a catalytically active protein foam.

2. The method according to claim 1, wherein, in order to produce a monoenzyme foam in the solution of step B), the catalytically active protein which is fused to the first connector is identical to the catalytically active protein which is fused to the second connector, or wherein, in order to produce a bi-enzyme foam, the catalytically active protein which is fused to the first connector is different from the catalytically active protein which is fused to the second connector.

3. The method according to claim 1, wherein, in order to produce a bi-enzyme foam using two orthogonal connector pairs, the solution of step B) contains a first catalytically active protein fused to a first connector, and a second catalytically active protein different from the first catalytically active protein, which is fused to a second connector, wherein the first connectors form the first connector pair and the second connectors form the second connector pair.

4. The method according to claim 1, wherein, in order to produce a multi-enzyme foam using at least three orthogonal connector pairs, the solution of step B) contains at least three different catalytically active proteins, each fused to a connector, wherein the respective connectors each form a connector pair.

5. The method according to claim 1, wherein the catalytically active proteins fused to the first connector and the catalytically active proteins fused to the second connector are heterologously expressed and the connectors are a genetically fused portion of the proteins.

6. The method according to claim 5, wherein the connectors are peptide tags.

7. The method according to claim 1, wherein the catalytically active proteins are expressed heterologously and the connectors are subsequently chemically bound to the catalytically active proteins.

8. The method according to claim 1, wherein the catalytically active protein foam is dried in a step E) after step D).

9. The method according to claim 8, wherein the catalytically active protein foam dried in step E) is used to convert at least one substrate to at least one product in the reaction chamber in a liquid flow.

10. The method according to claim 1, wherein the catalytically active proteins are enzymes.

11. The method according to claim 10, wherein the enzymes are selected from Lactobacillus brevis alcohol dehydrogenase (LbADH), Bacillus subtilis glucose 1-dehydro-genase (BsGDH), Enterobacter sp. phenacrylate decarboxylase (EsPAD), Saccharomyces cerevisiae NADP(H)-dependent alcohol dehydrogenase (Gre2P), Streptomnyces sp. (S)-imine reductase (GF3546), and Halomonas elongata amine transaminase (HEWT).

12. The method according to claim 1, wherein the bubbles produced in step B) have a diameter of 5-1500 µm and the pore size of the bubbles is between 10 and 2000 µm.

13. The method according to claim 1, wherein cross-linked protein thin films are present on the bubbles of the catalytically active protein foam produced in step D) and wherein the cross-linked protein thin films have a layer thickness of 0.1-200 µm.

14. The method according to claim 1, wherein the catalytically active protein foam is stabilized by additives.

15. The method according to claim 14, wherein a surfactant is used as the additive.

16. The method according to claim 1, wherein composite materials are added to the catalytically active protein foam.

17. The method according to claim 16, wherein the composite materials are selected from DNA-silica nanocomposite materials or DNA-silica nanocomposite materials, which additionally comprise carbon nanotubes.

18. A catalytically active protein foam produced by a method according to claim 1.

19. A method of biocatalysis, comprising providing the catalytically active protein foam according to claim 18, and performing biocatalysis using the catalytically active protein foam.

20. The method according to claim 19, wherein the catalytically active protein foam is used as a carrier-free, immobilized material for flow biocatalysis in a flow bioreactors.


SEQUENCE LISTING (15 sequences)

SEQ ID NO: 1 - DNA, 636 bp, Artificial Sequence
  Synthetic
atggctcacatcgttatggtggacgcttacaaaccgaccaaaggtggtggtggtagcaacaccttcgacaaacatgatctgagcggttttgttggtaaacatctggtgtatacctatgataatggctgggagtatgagatctatgtgaaaaatgaaaacaccctggattatcgcattcatagcggtctggttggtaatcgttgggttaaagatcagcaggcatatattgttcgtgtgggtgaaagcatctataaaatcagctggaccgaaccgaccggcaccgatgttagcctgattgttaatctgggtgatagcctgtttcatggcaccatcttttttccgcgttgggtgatgaataatccggaaaaaaccgtttgctttcagaacgatcatattccgctgatgaatagctatcgtgatgcaggtccggcatatccgaccgaagttattgatgaatttgccaccattacctttgttcgtgattgtggtgcaaataacgaaagcgttattgcatgtgcagcaagcgaactgccgaaaaactttccggataatctgaaaggtggtggtggtagcgcacatattgttatggttgatgcatataaaccgaccaaaggtcatcatcaccatcatcattaa

SEQ ID NO: 2 - DNA, 882 bp, Artificial Sequence
  Synthetic
atgaacaccttcgacaaacatgatctgagcggttttgttggtaaacatctggtgtatacctatgataatggctgggagtatgagatctatgtgaaaaatgaaaacaccctggattatcgcattcatagcggtctggttggtaatcgttgggttaaagatcagcaggcatatattgttcgtgtgggtgaaagcatctataaaatcagctggaccgaaccgaccggcaccgatgttagcctgattgttaatctgggtgatagcctgtttcatggcaccatcttttttccgcgttgggtgatgaataatccggaaaaaaccgtttgctttcagaacgatcatattccgctgatgaatagctatcgtgatgcaggtccggcatatccgaccgaagttattgatgaatttgccaccattacctttgttcgtgattgtggtgcaaataacgaaagcgttattgcatgtgcagcaagcgaactgccgaaaaactttccggataatctgaaaggtggtggtggtagcgttgataccctgagcggtctgagcagcgaacagggtcagagcggtgatatgaccattgaagaagatagcgcaacccacatcaaattcagcaaacgtgatgaagatggtaaagaactggcaggcgcaaccatggaactgcgtgatagcagcggtaaaaccattagcacctggattagtgatggtcaggtgaaagatttttatctgtaccctggcaaatacacctttgttgaaaccgcagcaccggatggttatgaagttgcaaccgcaattacctttaccgttaatgaacagggccaggttaccgtgaatggtaaagcaaccaaaggtgatgcacatattggtcatcatcaccatcatcattaa

SEQ ID NO: 3 - DNA, 1134 bp, Artificial Sequence
  Synthetic
atgcatcatcaccaccatcatggtgttgataccctgagcggtctgagcagcgaacagggtcagagcggtgatatgaccattgaagaagatagcgcaacccacatcaaattcagcaaacgtgatgaagatggtaaagaactggcaggcgcaaccatggaactgcgtgatagcagcggtaaaaccattagcacctggattagtgatggtcaggtgaaagatttttatctgtaccctggcaaatacacctttgttgaaaccgcagcaccggatggttatgaagttgcaaccgcaattacctttaccgttaatgaacagggccaggttaccgtgaatggtaaagcaaccaaaggtgatgcacatattggtggtggtggtagcagcaatcgcctggatggtaaagttgcaattattaccggtggcaccctgggtattggtctggcaattgcaaccaaatttgttgaagagggtgccaaagttatgattaccggtcgtcatagtgatgttggtgaaaaagcagcaaaaagcgttggtacaccggatcagattcagttttttcagcatgatagcagtgatgaagatggttggaccaaactgtttgatgcaaccgaaaaagcatttggtccggttagcaccctggttaataatgcaggtattgcagtgaataaaagcgtggaagaaaccaccaccgcagaatggcgtaaactgctggcagttaatctggatggtgttttttttggtacacgtctgggtattcagcgcatgaaaaacaaaggtctgggtgcaagcattatcaacatgagcagcattgaaggttttgttggtgatccgagcctgggtgcatataatgcaagcaaaggtgcagttcgtattatgagcaaaagcgcagcactggattgtgcactgaaagattatgatgttcgtgtgaataccgttcatccgggttatatcaaaacaccgctggttgatgatctgcctggtgccgaagaagcaatgagccagcgtaccaaaaccccgatgggtcatattggtgaaccgaatgatattgcctatatctgtgtttatctggccagcaacgaaagtaaatttgcaaccggtagcgaatttgttgtggatggtggttataccgcacagtaa

SEQ ID NO: 4 - DNA, 876 bp, Artificial Sequence
  Synthetic
atgtatccggatttaaaaggaaaagtcgtcgctattacaggagctgcttcagggctcggaaaggcaatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaagagttcggcacactcgatattatgattaataatgccggtcttgaaaatcctgtgccatctcacgaaatgccgcttaaggattgggataaagtcatcggcacaaacttaacaggtgcctttttaggaagccgtgaagcgattaaatatttcgtagaaaacgatatcaagggaaatgtcatcaacatgtccagtgtgcacgaagtgattccttggccgttatttgttcactacgcggcaagtaaaggcgggataaagctgatgacagaaacattggcgttggaatacgcgccgaagggcattcgcgtgaacaatatcgggccaggtgcgatcaatacgccaatcaatgctgaaaaatttgctgaccctaaacagaaagcagatgtagaaagcatgattccgatggggtatatcggcgaaccggaggagatcgccgcagtggcagtgtggcttgcttcgaaggaatccagctatgttacaggcatcacattgtttgcggacggcggaatgacgaaatatccttctttccaggcaggacgcggtggtggtggtggtagcggtggtggtggtagcgcccatattgttatggtggatgcatataaaccgaccaaaggtcatcatcaccatcaccattga

SEQ ID NO: 5 - DNA, 1164 bp, Artificial Sequence
  Synthetic
atggctcacatcgttatggttgacgcttacaaaccgaccaaaggtggtggtggtagctcagtttttgtttcaggtgctaacgggttcattgcccaacacattgtcgatctcctattgaaggaagactataaggtcatcggttctgctagaagtcaagaaaaggccgagaatttaacggaggcctttggtaacaatccaaacttctccatggaaattgtcccagacatatctaagctggacgcatttgaccatgttttccaaaagcacggcaaggagatcaagatagttctacatacggcctctccattctgctttgatatcactgacagtgaacgcgatttattaattcctgctgtgaacggtgttaagggaattctccactcaattaaaaaatacgccgctgattctgtagaacgtgtagttctcacctcttcttatgcagctgtgttcgacatggcaaaagaaaacgataaatctttaacatttaacgaagaatcctggaacccagcaacctgggagagttgccaaagtgacccggttagcgcctactgtggttctaagaagtttgctgaaaaagcagcttgggaatttctagaggagaatagagacgctgtaaaattcgaattaactgccgttaacccagtttacgtttttggtccgcaaatgtttgacaaagatgtgaaaaaacacttgaacacatcttgcgaactcgtcaacagcttgatgcatttatcaccagaggacaagataccggaactatttggtggatacattgatgttcgtgatgttgcaaaggctcatttagttgccttccaaaagagggaaacaattggtcaaagactaatcgtatcagaggccagatttactatgcaggatgttctcgatatccttaacgaagacttccctatcctaaaaggcaatattccagtggggaaaccaggttctggtgctacccataacacccttggtgctactcttgataataaaaagagtaagaaattgttaggtttcaagttcaggaacttgaaagagaccattgacgacactgcctcccaaattttaaaatttgagggcagaataggtggtggtggtagcgcccatattgttatggtggatgcatataaaccgaccaaaggtcatcatcaccatcaccattgagtttga

SEQ ID NO: 6 - DNA, 1176 bp, Artificial Sequence
  Synthetic
atgtatccggatttaaaaggaaaagtcgtcgctattacaggagctgcttcagggctcggaaaggcaatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaagagttcggcacactcgatattatgattaataatgccggtcttgaaaatcctgtgccatctcacgaaatgccgcttaaggattgggataaagtcatcggcacaaacttaacaggtgcctttttaggaagccgtgaagcgattaaatatttcgtagaaaacgatatcaagggaaatgtcatcaacatgtccagtgtgcacgaagtgattccttggccgttatttgttcactacgcggcaagtaaaggcgggataaagctgatgacagaaacattggcgttggaatacgcgccgaagggcattcgcgtgaacaatatcgggccaggtgcgatcaatacgccaatcaatgctgaaaaatttgctgaccctaaacagaaagcagatgtagaaagcatgattccgatggggtatatcggcgaaccggaggagatcgccgcagtggcagtgtggcttgcttcgaaggaatccagctatgttacaggcatcacattgtttgcggacggcggaatgacgaaatatccttctttccaggcaggacgcggtggtggtggtggtagcggtggtggtggtagcgttgataccctgagcggtctgagcagcgaacagggtcagagcggtgatatgaccattgaagaagatagcgcaacccacatcaaattcagcaaacgtgatgaagatggtaaagaactggcaggcgcaaccatggaactgcgtgatagcagcggtaaaaccattagcacctggattagtgatggtcaggtgaaagatttttatctgtaccctggcaaatacacctttgttgaaaccgcagcaccggatggttatgaagttgcaaccgcaattacctttaccgttaatgaacagggccaggttaccgtgaatggtaaagcaaccaaaggtgatgcacatattggtcatcatcaccatcaccattga

SEQ ID NO: 7 - DNA, 948 bp, Artificial Sequence
  Synthetic
atgcatcaccaccatcatcatggtgcccatattgttatggttgatgcctataaaccgaccaaaggtggtggtggtagcagcaaacagagcgttaccgttattggtctgggtccgatgggtcaagcaatggttaatacctttctggataatggtcatgaagtgaccgtttggaatcgtaccgcaagcaaagccgaagcactggttgcacgtggtgcagttctggcaccgaccgttgaagatgcactgagcgcaaatgaactgattgttctgagcctgaccgattatgatgcagtttatgcaattctggaaccggttaccggtagcctgagcggtaaagttattgcaaatctgagcagcgatacaccggataaagcacgtgaagcagcaaaatgggcagcaaaacatggtgcaaaacatctgaccggtggtgttcaggttccgcctccgctgattggtaaaccggaaagcagcacctattatagcggtccgaaagatgtttttgatgcccatgaagataccctgaaagttctgaccaatgcagattatcgtggtgaagatgccggtctggcagcaatgtattatcaggcacagatgaccattttttggaccaccatgctgagctattatcagaccctggcattaggtcaggcaaatggtgttagcgccaaagaactgctgccgtatgcaacaatgatgaccagcatgatgccgcattttctggaactgtatgcacagcatgttgatagtgccgattatccgggtgatgttgatcgtctggcaatgggtgcagcaagcgttgatcatgttctgcatacccatcaggatgccggtgttagcaccgttctgcctgcagcagttgcagaaatctttaaagcaggtatggaaaaaggctttgccgaaaatagctttagcagcctgattgaggttctgaaaaaaccggcagtttaa

SEQ ID NO: 8 - DNA, 1515 bp, Artificial Sequence
  Synthetic
atgcatcaccaccatcatcatggtgcccatattgttatggttgatgcctataaaccgaccaaaggtggtggtggtagtcagacccaggattatcaggcactggatcgtgcacatcatctgcatccgtttaccgattttaaagcactgggtgaagaaggtagtcgcgttgttacccatgccgaaggtgtttatatccatgatagcgaaggtaatcgtattctggatggtatggcaggtctgtggtgtgttaatttaggttatggtcgtcgtgaactggttgaagcagcaaccgcacagctggaacagctgccgtattataacacctttttcaaaaccacacatccgcctgcagttcgtctggcagaaaaactgtgtgatctggcaccggcacatattaatcgtgtgttttttaccggtagcggtagcgaagcaaatgataccgttctgcgtatggttcgtcgttattgggcactgaaaggtcagccggataaacagtggattattggtcgtgaaaatgcctatcatggtagcaccctggcaggtatgagcttaggtggcatggcaccgatgcatgcacaaggtggtccgtgtgttccgggtattgcacatattcgtcagccgtattggtttggtgaaggtcgtgatatgagtccggaagcatttggtcagacctgtgcagaagcactggaagaaaaaattctggaactgggcgaagaaaaagtggcagcatttattgcagaaccggtgcaaggtgccggtggtgcaattatgcctccggaaagctattggcctgccgttaaaaaagttctggccaaatatgatattctgctggttgccgatgaagtgatttgcggttttggtcgtttaggtgaatggtttggtagccagcattatggtctggaaccggatctgatgccgattgcaaaaggtctgagcagcggttatctgccgattggtggtgttctggttggtgatcgtgttgcagaaaccctgatcgaagaaggtggcgaatttttccatggttttacctatagcggtcatccgacctgtgccgcagttgcactgaaaaacctggaactgctggaagcggaaggtgttgtggatcgtgttcgtgatgatctgggtccgtatctggccgaacgttgggcaagcctggttgatcatccgattgttggtgaagcacgtagcctgggcctgatgggtgcactggaattagttgcagataaaaccaccggtcagcgttttgataaaagtttaggtgcaggtaatctgtgtcgtgatctgtgttttgccaatggtctggttatgcgtagcgttggcgataccatgattattagccctccgctggttattcgtcgcgaagaaattgatgaactggtggaactggcacgtcgtgccctggatgaaaccgcacgccagctgacccaggttccgcatacacaagaagaaccgaccgcaggtggtggtggtagtgcccatattgttatggttgatgcctataaaccgaccaaataa

SEQ ID NO: 9 - DNA, 1761 bp, Artificial Sequence
  Synthetic
atgcatcatcaccaccatcatggtgttgataccctgagcggtctgagcagcgaacagggtcagagcggtgatatgaccattgaagaagatagcgcaacccacatcaaattcagcaaacgtgatgaagatggtaaagaactggcaggcgcaaccatggaactgcgtgatagcagcggtaaaaccattagcacctggattagtgatggtcaggtgaaagatttttatctgtaccctggcaaatacacctttgttgaaaccgcagcaccggatggttatgaagttgcaaccgcaattacctttaccgttaatgaacagggccaggttaccgtgaatggtaaagcaaccaaaggtgatgcacatattggtggtggtggtagtcagacccaggattatcaggcactggatcgtgcacatcatctgcatccgtttaccgattttaaagcactgggtgaagaaggtagtcgcgttgttacccatgccgaaggtgtttatatccatgatagcgaaggtaatcgtattctggatggtatggcaggtctgtggtgtgttaatttaggttatggtcgtcgtgaactggttgaagcagcaaccgcacagctggaacagctgccgtattataacacctttttcaaaaccacacatccgcctgcagttcgtctggcagaaaaactgtgtgatctggcaccggcacatattaatcgtgtgttttttaccggtagcggtagcgaagcaaatgataccgttctgcgtatggttcgtcgttattgggcactgaaaggtcagccggataaacagtggattattggtcgtgaaaatgcctatcatggtagcaccctggcaggtatgagcttaggtggcatggcaccgatgcatgcacaaggtggtccgtgtgttccgggtattgcacatattcgtcagccgtattggtttggtgaaggtcgtgatatgagtccggaagcatttggtcagacctgtgcagaagcactggaagaaaaaattctggaactgggcgaagaaaaagtggcagcatttattgcagaaccggtgcaaggtgccggtggtgcaattatgcctccggaaagctattggcctgccgttaaaaaagttctggccaaatatgatattctgctggttgccgatgaagtgatttgcggttttggtcgtttaggtgaatggtttggtagccagcattatggtctggaaccggatctgatgccgattgcaaaaggtctgagcagcggttatctgccgattggtggtgttctggttggtgatcgtgttgcagaaaccctgatcgaagaaggtggcgaatttttccatggttttacctatagcggtcatccgacctgtgccgcagttgcactgaaaaacctggaactgctggaagcggaaggtgttgtggatcgtgttcgtgatgatctgggtccgtatctggccgaacgttgggcaagcctggttgatcatccgattgttggtgaagcacgtagcctgggcctgatgggtgcactggaattagttgcagataaaaccaccggtcagcgttttgataaaagtttaggtgcaggtaatctgtgtcgtgatctgtgttttgccaatggtctggttatgcgtagcgttggcgataccatgattattagccctccgctggttattcgtcgcgaagaaattgatgaactggtggaactggcacgtcgtgccctggatgaaaccgcacgccagctgacccaggttccgcatacacaagaagaaccgaccgcataa

SEQ ID NO: 10 - DNA, 966 bp, Artificial Sequence
  Synthetic
atgcatcatcaccaccatcatggtgcagaatatgttcgtgccctgtttgattttaatggcaacgatgaagaagatctgccgttcaaaaaaggtgatattctgcgtattcgtgacaaaccggaagaacagtggtggaatgcagaagatagcgaaggtaaacgtggtatgattccggttccgtatgtggaaaaatatggtggtggtggtagcagcaatcgcctggatggtaaagttgcaattattaccggtggcaccctgggtattggtctggcaattgcaaccaaatttgttgaagagggtgccaaagttatgattaccggtcgtcatagtgatgttggtgaaaaagcagcaaaaagcgttggtacaccggatcagattcagttttttcagcatgatagcagtgatgaagatggttggaccaaactgtttgatgcaaccgaaaaagcatttgctccggttagcaccctggttaataatgcaggtattgcagtgaataaaagcgtggaagaaaccaccaccgcagaatggcgtaaactgctggcagttaatctggatggtgttttttttggtacacgtctgggtattcagcgcatgaaaaacaaaggtctgggtgcaagcattatcaacatgagcagcattgaaggttttcttggtgatccgagcctgggtgcatataatgcaagcaaaggtgcagttcgtattatgagcaaaagcgcagcactggattgtgcactgaaagattatgatgttcgtgtgaataccgttcatccgggttatatcaaaacaccgctggttgatgatctgcctggtgcccaagaagcaatgagccagcgtaccaaaaccccgatgggtcatattggtgaaccgaatgatattgcctatatctgtgtttatctggccagcaacgaaagtaaatttgcaaccggtagcgaatttgttgtggatggtggttataccgcacagtaa

SEQ ID NO: 11 - DNA, 870 bp, Artificial Sequence
  Synthetic
atgtatccggatttaaaaggaaaagtcgtcgctattacaggagctgcttcagggctcggaaaggcaatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaagagttcggcacactcgatattatgattaataatgccggtcttgaaaatcctgtgccatctcacgaaatgccgcttaaggattgggataaagtcatcggcacaaacttaacaggtgcctttttaggaagccgtgaagcgattaaatatttcgtagaaaacgatatcaagggaaatgtcatcaacatgtccagtgtgcacgaagtgattccttggccgttatttgttcactacgcggcaagtaaaggcgggataaagctgatgacagaaacattggcgttggaatacgcgccgaagggcattcgcgtgaacaatatcgggccaggtgcgatcaatacgccaatcaatgctgaaaaatttgctgaccctaaacagaaagcagatgtagaaagcatgattccgatggggtatatcggcgaaccggaggagatcgccgcagtggcagtgtggcttgcttcgaaggaatccagctatgttacaggcatcacattgtttgcggacggcggaatgacgaaatatccttctttccaggcaggacgcggtggtggtggtggtagcggtggtggtggtagcccgccgccggcgctgccgccgaaacgtcgtcgtggtcatcatcaccatcaccattga

SEQ ID NO: 12 - DNA, 1080 bp, Artificial Sequence
  Synthetic
atgcatcatcaccaccatcatggtctgcagcgtcgtcgtgttaccgttcgtaaagcagatgccggtggtctgggtattagcattaaaggtggtcgtgaaaacaaaatgccgatcctgattagcaaaatctttaaaggtctggcagcagatcagaccgaagcactgtttgttggtgatgcaattctgagcgttaatggtgaagatctgagcagcgcaacccatgatgaagcagttcaggcactgaaaaaaaccggtaaagaagttgttctggaagtcaaatacatgaaagaagtgagcccgtactttaaaggtggtggtggtagcagcaatcgcctggatggtaaagttgcaattattaccggtggcaccctgggtattggtctggcaattgcaaccaaatttgttgaagagggtgccaaagttatgattaccggtcgtcatagtgatgttggtgaaaaagcagcaaaaagcgttggtacaccggatcagattcagttttttcagcatgatagcagtgatgaagatggttggaccaaactgtttgatgcaaccgaaaaagcatttggtccggttagcaccctggttaataatgcaggtattgcagtgaataaaagcgtggaagaaaccaccaccccagaatggcgtaaactgctggcagttaatctggatggtgttttttttggtacacgtctgggtattcagcgcatgaaaaacaaaggtctgggtgcaagcattatcaacatgagcagcattgaaggttttgttggtgatccgagcctgggtgcatataatgcaagcaaaggtgcagttcgtattatgagcaaaagcgcagcactggattgtgcactgaaagattatgatgttcgtgtgaataccgttcatccgggttatatcaaaacaccgctggttgatgatctgcctggtgccgaagaagcaatgagccagcgtaccaaaaccccgatgggtcatattggtgaaccgaatgatattgcctatatctgtgtttatctggccagcaacgaaagtaaatttgcaaccggtagcgaatttgttgtggatggtggttataccgcacagtaa

SEQ ID NO: 13 - DNA, 858 bp, Artificial Sequence
  Synthetic
atgtatccggatttaaaaggaaaagtcgtcgctattacaggagctgcttcagggctcggaaaggcaatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaagagttcggcacactcgatattatgattaataatgccggtcttgaaaatcctgtgccatctcacgaaatgccgcttaaggattgggataaagtcatcggcacaaacttaacaggtgcctttttaggaagccgtgaagcgattaaatatttcgtagaaaacgatatcaagggaaatgtcatcaacatgtccagtgtgcacgaagtgattccttggccgttatttgttcactacgcggcaagtaaaggcgggataaagctgatgacagaaacattggcgttggaatacgcgccgaagggcattcgcgtgaacaatatcgggccaggtgcgatcaatacgccaatcaatgctgaaaaatttgctgaccctaaacagaaagcagatgtagaaagcatgattccgatggggtatatcggcgaaccggaggagatcgccgcagtggcagtgtggcttgcttcgaaggaatccagctatgttacaggcatcacattgtttgcggacggcggaatgacgaaatatccttctttccaggcaggacgcggtggtggtggtggtagcggtggtggtggtagcggtgttaaagaatctctggttggtcatcatcaccatcaccattga

SEQ ID NO: 14 - DNA, 1032 bp, Artificial Sequence
  Synthetic
atgcatcatcaccaccatcatggtaccaaagcagatattggcaccccgagcaattttcagcatattggtcatgttggttgggatccgaataccggttttgatctgaataatctggatccggaactgaaaaacctgtttgatatgtgtggtattagcgaagcacagctgaaagatcgtgaaaccagcaaagtgatctatgactttatcgaaaaaaccggtggtgttgaagccgtgaaaaatgaactgcgtcgtcaggcaccgggtggtggtggtagcagcaatcgcctggatcgtaaagttgcaattattaccggtggcaccctgggtattggtctggcaattgcaaccaaatttgttgaagagggtgccaaagttatgattaccggtcgtcatagtgatgttggtgaaaaagcagcaaaaagcgttggtacaccggatcagattcagttttttcagcatgatagcagtgatgaagatggttggaccaaactgtttgatgcaaccgaaaaagcatttggtccggttagcaccctggttaataatgcaggtattgcagtgaataaaagcgtggaagaaaccaccaccgcagaatggcgtaaactgctggcagttaatctggatggtgttttttttggtacacgtctgggtattcagcgcatgaaaaacaaaggtctgggtgcaagcattatcaacatgagcagcattgaaggttttgttggtgatccgagcctgggtgcatataatgcaagcaaaggtgcagttcgtattatgagcaaaagcgcagcactggattgtgcactgaaagattatgatgttcgtgtgaataccgttcatccgggttatatcaaaacaccgctggttgatgatctgcctggtgccgaagaagcaatgagccagcgtaccaaaaccccgatgggtcatattggtgaaccgaatgatattgcctatatctgtgtttatctggccagcaacgaaagtaaatttgcaaccggtagcgaatttgttgtggatggtggttataccgcacagtaa

SEQ ID NO: 15 - DNA, 933 bp, Artificial Sequence
  Synthetic
atgtatccggatttaaaaggaaaagtcgtcgctattacaggagctgcttcagggctcggaaaggcaatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaagagttcggcacactcgatattatgattaataatgccggtcttgaaaatcctgtgccatctcacgaaatgccgcttaaggattgggataaagtcatcggcacaaacttaacaggtgcctttttaggaagccgtgaagcgattaaatatttcgtagaaaacgatatcaagggaaatgtcatcaacatgtccagtgtgcacgaagtgattccttggccgttatttgttcactacgcggcaagtaaaggcgggataaagctgatgacagaaacattggcgttggaatacgcgccgaagggcattcgcgtgaacaatatcgggccaggtgcgatcaatacgccaatcaatgctgaaaaatttgctgaccctaaacagaaagcagatgtagaaagcatgattccgatcgggtatatcggcgaaccggaggagatcgccgcagtggcagtgtggcttgcttcgaaggaatccagctatgttacaggcatcacattgtttgcggacggcggaatgacgaaatatccttctttccaggcaggacgcggtggtggtggtggtagcggtggtggtggtagcctggttggtgcactgatgcatgttatgcagaaacgtagccgtgcaattcatagcagtgatgaaggtgaagatcaagccggtgatgaagatgaggatggtcatcatcaccatcaccattga